(19) United States
(12) Patent Application Publication
(10) Pub. No.: US 2016/0106656 A1
(43) Pub. Date: Apr. 21, 2016

(54) Cosmetic Composition and Conditioning Agent

(71) Applicant: The Procter & Gamble Company, Cincinnati, OH (US)

(72) Inventors: Nobuaki UEHARA, Singapore (SG); Shoya YODA, Yokohama (JP); Akira ISHIKUBO, Tokyo (JP)

(21) Appl. No.: 14/518,530

(22) Filed: Oct. 20, 2014

(51) Int. Cl. A61K 8/81; A61Q 5/12

(52) U.S. Cl. CPC A61K 8/8158; A61K 8/8147; A61K 8/8152; A61Q 5/12

(57) ABSTRACT

Disclosed is a cosmetic composition comprising: a copolymer (α) or (β); a cationic surfactant; and a higher alcohol. The copolymer (α) includes a constituent unit derived from a monomer (a) acrylic acid and/or methacrylic acid, and at least one constituent unit derived from three different monomers, wherein from 50 to 100 mass % of a constituent unit derived from a vinyl monomer (A) having a carboxyl group. The copolymer (β) includes a constituent unit derived from a monomer (a) acrylic acid and/or methacrylic acid and at least one constituent unit derived from four different monomers, wherein from 50 to 100 mass % of a constituent unit derived from a vinyl monomer (A) having a carboxyl group and wherein from 50 to 100 mol % of the carboxyl groups are not neutralized.

### FIELD OF THE INVENTION

The present invention relates to a cosmetic composition having an extremely effect of heightening hair-conditioning effects, and to a conditioning agent comprising said cosmetic composition.

### BACKGROUND OF THE INVENTION

In recent years, consumers have been showing increased concern for damage to the hair, as shown by the vigorous market for damaged hair care products such as high-end shampoos, rinses, and outbath treatments. Rinses (conditioners) are used after washing the hair with shampoo in order to reduce damaged feel in hair (i.e., in order to improve texture). If a damaged feel still remains, there are treatments for use both in and outside the bathroom that can be utilized.

A damaged feel in hair manifests as coarse/rough feeling, hardness, or other sensory determiners caused by friction between individuals hairs or between the hair and the hand. In order to suppress this sort of damaged feel, rinses and treatments contain cationic surfactants such as stearyl trimethyl ammonium chloride as ingredients (softening ingredients) for reducing friction and softening the hair.

Meanwhile, in order to reduce damaged feel in hair, modifications to surfactant structure, the inclusion of a silicone oil, the inclusion of resins, and the like have been attempted. In cases in which a resin is included, it has been shown (for example, see patent document 1) that a cosmetic containing, for example, a copolymer comprising a unit having an anionic group and a unit having a silicon atom or a resin compound having an anionic group and a silicon atom within the molecule thereof can impart body to the hair, as well as superior conditioning effects such as softness and sleekness. It has also been shown (for example, see patent documents 2 and 3) that a cosmetic containing a resin compound comprising an anionic vinyl monomer and a nonionic vinyl monomer comprising a hydroxyl group will heighten conditioning power.

### PRIOR ART DOCUMENTS

### [Patent Document 1] Japanese Unexamined Patent Application Publication No. 2004-018414

### [Patent Document 2] Japanese Unexamined Patent Application Publication No. 2007-161986

### [Patent Document 3] Japanese Unexamined Patent Application Publication No. 2007-137830

In recent years, there has come to be a demand for conditioning effects even greater than those offered by the inventions disclosed in patent documents 1-3. In particular, there is an increased demand for selective restoration of highly damaged portion such as hair tips, and further improvements such as a 1 reduced coarse/rough feeling of hair after drying and smoothness of finger combing. Additionally, the ability to obtain high treatment effects from shampooing and conditioning, instead of using countless hair treatment products, would also enable the expenses borne by consumers to be reduced. Thus, there is an increased demand for improvements in such hair cosmetics.

Meanwhile, wash-out agents, such as rinses and treatments, must be rinsed off after being applied to the hair. However, investigation by the inventors has discovered the problem that many of the softening ingredients contained in such agents are washed away during such rinsing, leading to insufficient reduction in damaged feel.

An object of the present invention is to provide a cosmetic composition, a hair cosmetic, and a conditioning agent that solve the problem described above, do not exhibit losses in smoothness or other types of texture during use, and yield superior conditioning effects such as reduced coarse/rough feeling of hair after drying and smoothness of finger combing.

None of the existing art provides all of the advantages and benefits of the present invention.

### SUMMARY OF THE INVENTION

As described above, the problem has been discovered that rinses must be rinsed out with water after being applied to the hair, leading to many of the softening ingredients being washed away during rinsing. As the result of diligent research on the part of the inventors on the premise that enabling large amounts of softening ingredients such as cationic surfactants and conditioning ingredients such as silicone oils to remain on the hair even after rinsing would promise an improvement in conditioning ability, it was discovered that a copolymer comprising a specific proportion of a constituent unit derived from a specific monomer and having a specific weight average molecular weight imparts high conditioning effects in terms of reduced coarse/rough feeling of hair after drying and smoothness of finger combing without negatively affecting sleekness and other aspects of texture during use.

The present invention is directed to the following (hereinafter referred to as a “first aspect of the present invention”):

A cosmetic composition comprising:
from 0.05 to 5 mass % of a copolymer (α);
from 0.1 to 20 mass % of a cationic surfactant; and
from 0.1 to 20 mass % of a higher alcohol;
wherein the copolymer (α) includes a constituent unit derived from a monomer (a) below and at least one constituent unit derived from monomers (b) to (d) below;
wherein the copolymer (α) comprises from 1 to 99 mass % of the constituent unit derived from the monomer (a) below per a total mass of the copolymer (α), and from 50 to 100 mass % of the constituent unit derived from vinyl monomer (A) having a carboxyl group;
and wherein the copolymer (α) has a mass average molecular weight of from 3,000 to 400,000:
wherein the Monomer (a) is acrylic acid and/or methacrylic acid;
wherein the Monomer (b) is one or two or more types of vinyl monomers expressed by formulas (1), (2), and (3) below:

CH₂═C(R¹)—CO—(O—(CH₂)ₘ—CO)ₙ—OH (1)

(in formula (1), R¹ is a hydrogen atom or a methyl group, “m” is an integer from 1 to 4, and “n” is an integer from 1 to 4):

CH₂═C(R²)—COO—(CH₂)ₚ—OCO—(CH₂)_{q}—COOH (2)

(in formula (2), R² is a hydrogen atom or a methyl group, and “p” and “q” are each independently an integer from 2 to 6):

CH₂═C(R³)—COO—(CH₂)ₑ—OCO—X—COOH (3)

(in formula (3), R³ is a hydrogen atom or a methyl group, “e” is an integer from 1 to 4, and “X” is a cyclohexylene group optionally having a substituent or a phenylene group optionally having a substituent);

wherein the Monomer (c) is a vinyl monomer expressed by formula (4) below:

CH₂═C(R⁴)—CO—Y—(Z—O)ᵣ—R⁵ (4)

(in formula (4), R⁴ is a hydrogen atom or a methyl group, R⁵ is a hydrogen atom or an alkyl group having from 1 to 5 carbons, “Y” is an oxygen atom or NH, “Z” is an alkylene group having from 2 to 4 carbons and optionally having a substituent, and “r” is an integer from 1 to 15); and

wherein the Monomer (d) is a vinyl monomer expressed by formula (5) below:

CH₂═C(R⁶)—COO—R⁷ (5)

(in formula (5), R⁶ is a hydrogen atom or a methyl group, R⁷ is a straight chain or branched alkyl group having from 12 to 22 carbons, a cyclohexyl group optionally having a substituent, or a phenyl group optionally having a substituent).

The present invention is also directed to the following (hereinafter referred to as a “second aspect of the present invention”):

A cosmetic composition comprising:

from 0.05 to 5 mass % of a copolymer (β);
from 0.1 to 20 mass % of a cationic surfactant; and
from 0.1 to 20 mass % of a higher alcohol;
wherein the copolymer (β) includes a constituent unit derived from a monomer (a) below and at least one constituent unit derived from monomers (b) to (e) below;
wherein the copolymer (β) comprises from 50 to 100 mass % of the constituent unit derived from vinyl monomer (A) having a carboxyl group per a total mass of the copolymer (β), and wherein from 50 to 100 mol % of the carboxyl groups are not neutralized;
and wherein the copolymer (β) has a mass average molecular weight of from 3,000 to 400,000:
wherein the Monomer (a) is acrylic acid and/or methacrylic acid:
wherein the Monomer (b) is one or two or more types of vinyl monomers expressed by formulas (1), (2), and (3) below:

CH₂═C(R¹)—CO—(O—(CH₂)ₘ—CO)ₙ—OH (1)

(in formula (1), R¹ is a hydrogen atom or a methyl group, “m” is an integer from 1 to 4, and “n” is an integer from 1 to 4);

CH₂═C(R²)—COO—(CH₂)ₚ—OCO—(CH₂)_{q}—COOH (2)

(in formula (2), R² is a hydrogen atom or a methyl group, and “p” and “q” are each independently an integer from 2 to 6);

CH₂═C(R³)—COO—(CH₂)ₑ—OCO—X—COOH (3)

(in formula (3), R³ is a hydrogen atom or a methyl group, “e” is an integer from 1 to 4, and “X” is a cyclohexylene group optionally having a substituent or a phenylene group optionally having a substituent);

wherein the Monomer (c): a vinyl monomer expressed by formula (4) below:

CH₂═C(R⁴)—CO—Y—(Z—O)ᵣ—R⁵ (4)

(in formula (4), R⁴ is a hydrogen atom or a methyl group, R⁵ is a hydrogen atom or an alkyl group having from 1 to 5 carbons, “Y” is an oxygen atom or NH, “Z” is an alkylene group having from 2 to 4 carbons and optionally having a substituent, and “r” is an integer from 1 to 15);

wherein the Monomer (d): a vinyl monomer expressed by formula (5) below:

CH₂═C(R⁶)—COO—R⁷ (5)

(in formula (5), R⁶ is a hydrogen atom or a methyl group, R⁷ is a straight chain or branched alkyl group having from 12 to 22 carbons, a cyclohexyl group optionally having a substituent, or a phenyl group optionally having a substituent), and

wherein the Monomer (e): a vinyl monomer expressed by formula (6) below:

CH₂═C(R⁸)—CO—N(R⁹)R¹⁰ (6)

(in formula (6), R₈ is a hydrogen atom or a methyl group, R₉ and R₁₀ are each independently a hydrogen atom or a straight chain or branched alkyl group having from 1 to 4 carbons).

The cosmetic composition according to the first aspect of the present invention and the cosmetic composition according to the second aspect of the present invention may at times be collectively referred to as the “cosmetic composition according to the present invention”.

The cosmetic composition according to the present invention has superior conditioning effects, such as smoothness during application, reduced coarse/rough feeling of hair after drying, and smoothness of finger combing. If a silicone oil is also used, the adsorption of the silicone oil can be increased, enabling even greater conditioning effects to be imparted. In particular, a hair cosmetic having superior conditions effects can be obtained, making the cosmetic composition especially useful as a conditioning agent.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Embodiments of the present invention are described below in detail, but the following descriptions are of typical examples of embodiments of the present invention, and the present invention is in no way limited thereto. In the present specification, the expression “(meth)acrylic acid” encompasses both “acrylic acid” and “methacrylic acid”. Furthermore, in the present specification, the expression “˜” is used in a sense inclusive of the preceding and following numbers or physical values. Furthermore, in the present specification, “smoothness during application” refers to an evaluation given in the following working examples, and such effect will be considered inferior if the smoothness during application is worse than that of a “comparative standard product” even if reduced coarse/rough feeling of hair after drying, smoothness of finger combing, and cosmetic composition viscosity are superior.

### [Copolymers (α) and (β)]

The copolymer (α) used in the cosmetic composition according to the first aspect of the present invention is a copolymer (α) having a constituent unit derived from a vinyl monomer (A) having a carboxyl group, the proportion of the constituent unit derived from the vinyl monomer (A) with respect to the total mass of the copolymer (α) being from 50 to 100 mass %; the copolymer (α) comprising from 1 to 99 mass % of at least a constituent unit derived from a monomer (a) described below with respect to the total mass of the copolymer (α) as the constituent unit derived from the vinyl monomer (A), having a weight average molecular weight of from 3,000 to 400.000, and comprising at least one constituent unit derived from monomers (b) to (d) described below.

The copolymer (β) used in the cosmetic composition according to the second aspect of the present invention is a copolymer (β) including a constituent unit derived from a monomer (a) below and at least one constituent unit derived from monomers (b) to (e) below; the copolymer (β) comprising not less than 50 mass % and not more than 100 mass % of the constituent unit derived from the vinyl monomer (A) having a carboxyl group with respect to the total mass of the copolymer (β), and having a mass average molecular weight of from 3.000 to 400,000; from 50 to 100 mol % of the carboxyl groups therein not being neutralized.

<Constituent Unit Derived from the Vinyl Monomer (A)>

The copolymer (α) and copolymer (β) used in the present invention each have a constituent unit derived from the vinyl monomer (A) having a carboxyl group, the proportion of said constituent unit derived from the vinyl monomer (A) being from 50 to 100 mass % with respect to the total masses of the copolymer (α) and the copolymer (β), respectively.

The copolymer (α) and copolymer (β) used in the present invention must each comprise a specific proportion of the constituent unit derived from the vinyl monomer (A) having a carboxyl group for the following reasons.

One of the problems to be solved by the present invention is to increase the amount of conditioning ingredients adsorbed to damaged hair, heightening conditioning effects. The copolymers used in the present invention are designed so as to enhance interaction with the cationic surfactant, and the following two points can be suggested as important points of the presumed mechanism thereof,

1) Interaction between the copolymer and a cationic surfactant: The interaction between the copolymer and the cationic surfactant is enhanced by the polarization of the oxygen In the —OH of the carboxyl group in the copolymer.

2) Interaction between hair and a composite of the copolymer and the cationic surfactant: The interaction between the (anionic) hair surface and the polarized hydrogen atoms of the carboxyl group of the copolymer and the cationic group of the cationic surfactant is enhanced.

Investigation by the inventors revealed that having the proportion of the constituent unit derived from the vinyl monomer (A) be from 50 to 100 mass % of the total masses of the copolymer (α) and the copolymer (β), respectively, enhances the effects of the presumed mechanism (the above 1) and 2)), yielding satisfactory conditioning effects in terms of smoothness during application, reduced coarse/rough feeling of hair after drying, smoothness of finer combing after drying, hair manageability after drying, and the like. From considerations of achieving balanced enhancement of these various conditioning effects, the proportion of the constituent unit derived from, the vinyl monomer (A) to the total masses of the copolymer (α) and the copolymer (β), respectively, is preferably at least 55 mass %, more preferably greater than 60 still more preferably at least 65 mass %, and especially preferably at least 70 mass %; and preferably no more than 99 mass %, more preferably no more than 90 mass %.

As described above, in the copolymer (α) and copolymer (fi) used in the present invention, the technical significance of the constituent unit derived from the vinyl monomer (A) is that the constituent unit derived from the vinyl monomer (A) provides a carboxyl group for interacting with the hair surface and the cationic surfactant. Therefore, there is no particular limitation upon the type of constituent unit derived from the vinyl monomer (A) apart from the monomer (a) as long as it has a carboxyl group; specific examples include crotonic acid, maleic acid, fumaric acid, itaconic acid, angelic acid, tiglic acid, 2-(meth)acryoyl oxyethyl succinate, 2-carboxyethyl (meth)acrylate, 2(2-carboxyethoxy)ethyl (meth)acrylate, 2-(meth)acryoyl oxyethyl hexahydrophthalate, and terephthalate (meth)acrylate. Of these, (meth)acrylic acid or an unsaturated carboxylic monomer typified by the compounds represented in formulas (1), (2), and (3) shown below is preferable. The unsaturated carboxylic acid monomer preferably has from 3 to 22 carbons, more preferably 4 or more carbons; and preferably 20 or fewer carbons, more preferably 18 or fewer carbons, even more preferably 10 or fewer carbons, and especially preferably 6 or fewer carbons. Furthermore, the number of carboxyl groups in the constituent unit derived from the vinyl monomer (A) is preferably from 1 to 4, more preferably from 1 to 3, even more preferably from 1 to 2, and most preferably 1.

<Constituent Unit Derived from Monomer (a)>

in the copolymer (α) and copolymer (β) used in the present invention, the vinyl monomer (A) comprises the following monomer (a) in order to enhance interaction with the cationic surfactant.

Monomer (a): acrylic acid and/or methacrylic acid.

The monomer (a) has a large amount of carboxyl groups with respect to its mass; therefore, the fact that the copolymer (α) and the copolymer (β) used in the present invention each comprise a constituent unit derived from the monomer (a) allows satisfactory conditioning effects, which constitute the base of a conditioning polymer, to be obtained. In addition, including the monomer (a) and having the amount of carboxyl groups within the copolymer be at or above a constant amount allows conditioning effects to be obtained. The monomer (a) is also advantageous in that it is generally easily obtainable.

A composite formed with acrylic acid or a cationic surfactant is preferably used as the monomer (a) from considerations of even dissolution or dispersion within the composition. Meanwhile, methacrylic acid is preferable because it enables the copolymer (α) and the copolymer (β) to be imparted with hydrophobic properties, and can further contribute to reduced coarse/rough feeling of hair after drying. The copolymer (α) and the copolymer (β) used in the present invention may each comprise acrylic acid alone as the monomer (a), or may comprise acrylic acid and methacrylic acid. Of these options, comprising acrylic acid and methacrylic acid is preferable. In such cases, the proportion acrylic acid to the total mass of the monomer (a) is preferably from 50 to 99 mass %.

The copolymer (α) used in the present invention comprises from 1 to 99 mass % of the constituent unit derived from the monomer (a) with respect to the total mass of the copolymer (α). Having the amount of the constituent unit derived from the monomer (a) comprised by the copolymer (α) be at least the lower limit described above is preferable because such an amount will enhance the effects obtained via the inclusion of the acrylic acid and/or methacrylic acid, and said amount is preferably at least 10 mass % with respect to the total mass of the copolymer (α), more preferably at least 30 mass %, still more preferably at least 50 mass %, especially preferably at least 60 mass %, and most preferably at least 65 mass %. Meanwhile, having said amount be no more than the upper limit described above is preferable because the hydrophilic monomer and/or hydrophobic monomer other than the monomer (a) will be copolymerized, thereby enhancing the affinity thereof with damaged hair, and texture after drying can be improved; and the amount of constituent unit derived from the monomer (a) is preferably no more than 95 mass %, more preferably no more than 90 mass %, and still more preferably no more than 80 mass %.

The copolymer (β) used in the present invention preferably comprises from 1 to 99 of the constituent unit derived from the monomer (a) with respect to the total mass of the copolymer (β). Having the amount of the constituent unit derived from the monomer (a) comprised by the copolymer (β) be at least the lower limit described is preferable because such an amount will enhance the effects obtained via the inclusion of the acrylic acid and/or methacrylic acid, and said amount is more preferably at least 10 mass %, still more preferably at least 30 mass %, even more preferably at least 50 mass %, especially preferably at least 60 mass %, and most preferably at least 65 mass %. Meanwhile, having said amount be no more than the upper limit described above is preferable because the hydrophilic monomer and/or hydrophobic monomer other than the monomer (a) will be copolymerized, thereby enhancing the affinity thereof with damaged hair, and texture after drying can be improved; and the amount of constituent unit derived from the monomer (a) is more preferably no more than 95 mass %, still more preferably no more than 90 mass %, and especially preferably no more than 80 mass %

<Constituent Unit Derived from Monomer (b)>

From considerations of the fact that imparting the copolymer (α) and the copolymer (β) with hydrophilic properties allows the affinity thereof to damaged hair to be increased, and from considerations of the fact that imparting the copolymers with hydrophobicity will allow for improved texture after drying, the copolymer (α) and the copolymer (β) used in the present invention preferably comprise the following constituent unit derived from monomer (b) as a constituent unit derived from the vinyl monomer (A) along with the monomer (a).

Monomer (b): one or two or more types of vinyl monomers expressed by formulas (1), (2), and (3) below:

CH₂C(R¹)—CO—(O—(CH₂)ₘ—CO)ₙ—OH (1)

(in formula (1), R¹ is a hydrogen atom or a methyl group, “m” is an integer from 1 to 4, and “n” is an integer from 1 to 4);

CH₂═C(R²)—COO—(CH₂)ₚ—OCO—(CH₂)_{q}—COOH (2)

(in formula (2), R² is a hydrogen atom or a methyl group, and “p” and “q” are each independently an integer from 2 to 6); and

CH₂═C(R³)—COO—(CH₂)ₑ—OCO—X—COOH (3)

(in formula (3), R³ is a hydrogen atom or a methyl group, “e” is an integer from 1 to 4, and “X” is a cyclohexylene group optionally having a substituent or a phenylene group optionally having a substituent).

In formula (1), R¹ is preferably a hydrogen atom, m is preferably 2 or 3. n is preferably from 1 to 2, most preferably 1.

Specific examples of the vinyl monomer represented by formula (1) above include 2-carboxyethyl (meth)acrylate, 2(2-carboxyethoxy)ethyl (meth)acrylate, and the like.

In formula (2), R² is preferably a hydrogen atom, p is preferably 2 or 3, and q is preferably 2 or 3.

A specific example of the vinyl monomer represented by formula (2) is 2-(meth)acryoyl oxyethyl succinate.

In formula (3), R³ is preferably a methyl group, e is preferably an integer from 1 to 3, most preferably 2. The substituent of the cyclohexylene group optionally having a substituent or the phenylene group optionally having a substituent represented by “X” is preferably a straight-chain or branched alkyl group having from 1 to 4 carbons, more preferably a straight-chain or branched alkyl group having from 1 to 3 carbons. “X” is preferably a phenylene group or cyclohexylene group not having a substituent, most preferably a cyclohexylene group.

Specific examples of the vinyl monomer represented by formula (3) include (meth)acrylate ethyl terephthalate, (meth)acrylate propyl terephthalate, 2-(meth)acryoyl oxyethyl hexahydrophthalate, and the like.

The compounds represented by formula (1) or (2) is preferable as a monomer (b) for imparting hydrophilic properties. Using a monomer (b) having these hydrophilic groups allows the copolymer to adsorb more easily to damaged hair, yielding a smoothness of finger combing. The compound represented by formula (3) is preferable as a monomer (b) for imparting hydrophobic properties. Using a monomer (b) having this hydrophobic group allows the effects of improving the texture of the hair itself to which the copolymer is adsorbed and reducing hair coarse/rough feeling to be obtained.

One type of constituent unit derived from monomer (b) may be used in isolation in the copolymer (α) and copolymer (β) used in the present invention, or two or more may be used in combination; or one of the compounds shown in formulas (1), (2), and (3) may additionally be used.

The amount of die constituent unit derived from monomer (b) comprised by the copolymer (α) and copolymer (β) used in the present invention, respectively, is preferably from, 1 to 99 mass %. Having said amount be at least the lower limit described above is preferable because the effects described above will be enhanced, said amount preferably being at least 10 mass %, more preferably at least 20 mass %, especially preferably at least 30 mass %, and most preferably at least 40 mass %. Meanwhile, from considerations of the fact that, if said amount is no more than the upper limit described above, the amount of carboxyl groups with respect to the unit mass will be increased by the constituent unit derived from the monomer (a), said amount is preferably no more than 90 mass %, more preferably no more than 80 mass %, and still more preferably no more than 60 mass %.

<Constituent Unit Derived from Monomer (c)>

The copolymer (α) and copolymer (β) used in the present invention preferably comprise the constituent unit derived from monomer (c) shown in formula (4) below, as this will allow a suitable degree of hydrophilic properties to be imparted to the copolymer (α). Having a suitable amount of hydrophilic properties allows for affinity to damaged hair to be increased without negatively affecting smoothing during use and other aspect of texture, and lets the composition adsorb more easily to damaged hair. This allows for a smoothness of finger combing to be obtained,

CH₂═C(R⁴)—CO—Y—(Z—O)ᵣ—R⁵ (4)

(in formula (4), R⁴ is a hydrogen atom or a methyl group, R⁵ is a hydrogen atom or an alkyl group having from 1 to 5 carbons, “Y” is an oxygen atom or NH, “Z” is an alkylene group having from 2 to 4 carbons and optionally having a substituent, and “r” is an integer from 1 to 15); and

In formula (4), R⁴ is preferably a hydrogen atom. Because the monomer (c) preferably has a suitable level of hydrophilicity, R⁵ is preferably a hydrogen atom or an alkyl group having from 1 to 3 carbons, and more preferably a hydrogen atom or an alkyl group having from 1 or 2 “Y” preferably represents an oxygen atom, “r” is preferably at least 2, and preferably no more than 10, more preferably no more than 6, and still more preferably no more than 3.

If the alkylene group represented by “Z” in formula (4) has a substituent, such substituent is one that is not reactive with the other parts of the copolymer (α) and copolymer (β) used in the present invention. If the volume occupied by said substituent is too great, the volume occupied by the substituent with respect to the structural portion Y—(Z—O)ᵣ—R⁵ of the monomer (c), thereby weakening the interaction between the carboxyl group of the vinyl monomer (A) and the cationic surfactant and potentially reducing the level of conditioning ingredient adsorption. Therefore, the substituent preferably has a molecular weight of no more than 50, with specific examples including a hydroxyl group, a methoxy group, an ethoxy group, an alkyl group, a hydroxyalkyl group, a methoxyalkyl group, an ethoxyalkyl group, or the like. The substituent of the alkylene group represented by “Z” preferably has a lower molecular weight than the structural portion —(Z—O)ᵣ—. However, “Z” more preferably does not have a substituent. “Z” is preferably an alkylene group having 2 or 3 carbons, most preferably an ethylene group having 2 carbons.

Examples of the monomer (c) represented by formula (4) include methoxy polyethylene glycol (meth)acrylate (the number of polyethylene glycol repetitions (“r” in formula (4)) being from 2 to 15), polyethylene glycol (meth)acrylate (the number of polyethylene glycol repetitions (“r” in formula (4)) being from 2 to 15), methoxy polyethylene glycol/polypropylene glycol (meth)acrylate (the number of polyethylene glycol/polypropylene glycol repetitions (“r” in formula (4)) being from 2 to 15), polyethylene glycol/polypropylene glycol (meth)acrylate (the number of polyethylene glycol/polypropylene glycol repetitions (“r” in formula (4)) being from 2 to 15), methoxy polyethylene glycol/polybutylene glycol (meth)acrylate (the number of polyethylene glycol/polybutylene glycol repetitions (“r” in formula (4)) being from 2 to 15), polyethylene glycol/polybutylene glycol(meth)acrylate (the number of polyethylene glycol/polybutylene glycol repetitions (“r” in formula (4)) being from 2 to 15), methoxy polyethylene glycol (meth)acrylamide (the number of polyethylene glycol repetitions (“r” in formula (4)) being from 2 to 15), polyethylene glycol (meth)acrylamide (the number of polyethylene glycol repetitions (“r” in formula (4)) being from 2 to 15), 2-hydroxyethyl(meth)acrylate, 3-hydroxypropyl(meth)acrylate, 2,3-dihydroxypropyl(meth)acrylate, and 2-hydroxyethyl(meth)acrylamide; preferably methoxy polyethylene glycol (meth)acrylate (the number of polyethylene glycol repetitions (“r” in formula (4)) being from 2 to 10), polyethylene glycol (meth)acrylate (the number of polyethylene glycol repetitions (“r” in formula (4)) being from 2 to 10), methoxy polyethylene glycol/polypropylene glycol (meth)acrylate (the number of polyethylene glycol/polypropylene glycol repetitions (“r” in formula (4)) being from 2 to 10), polyethylene glycol/polypropylene glycol (meth)acrylate (the number of polyethylene glycol/polypropylene glycol repetitions (“r” in formula (4)) being from 2 to 10), methoxy polyethylene glycol/polybutylene glycol (meth)acrylate (the number of polyethylene glycol/polybutylene glycol repetitions (“r” in formula (4)) being from 2 to 10), polyethylene glycol/polybutylene glycol(meth)acrylate (the number of polyethylene glycol/polybutylene glycol repetitions (“r” in formula (4)) being from 2 to 10), 2-hydroxyethyl(meth)acrylate, 2-hydroxyethyl(meth)acrylate, 3-hydroxypropyl(meth)acrylate, 2,3-dihydroxypropyl(meth)acrylate, and 2-hydroxyethyl (meth)acrylamide; and more preferably methoxy polyethylene glycol (meth)acrylate (the number of polyethylene glycol repetitions (“r” in formula (4)) being from 2 to 10) and polyethylene glycol (meth)acrylate (the number of polyethylene glycol repetitions (“r” in formula (4)) being from 2 to 10),

One type of constituent unit derived from monomer (c) may be used in isolation in the copolymer (α) and copolymer (β) used in the present invention, or two or more types may be used in combination.

The proportion of constituent unit derived from monomer (c) in the copolymer (α) and copolymer (β) used in the present invention is preferably from 1 to 50 mass % with respect to the total masses of the copolymer (α) and the copolymer (β). Having the proportion of constituent unit derived from monomer (c) be at least 1 mass % allows sufficient conditioning ingredient adsorption to be obtained due to the increased affinity to damaged hair, and having said proportion be no more than 50 mass % ensures the proportion of the constituent unit derived from the vinyl monomer (A) in the copolymer (α) and the copolymer (β), allowing conditioning ingredient adsorption to be increased. From considerations of enhancing these effects, the proportion of the constituent unit derived from monomer (c) in each copolymer (α) is more preferably at least 3 mass %, still more preferably at least 5 mass %, and especially more preferably at least 10 mass %; and more preferably no more than 40 mass %, especially preferably no more than 30 mass %.

<Constituent Unit Derived from Monomer (d)>

The hydrophobic properties of the copolymer (α) and the copolymer (β) can be increased by having the copolymer (α) and copolymer (β) used in the present invention comprise the monomer (d) shown in formula (5) below as a constituent unit. In particular, the use of a monomer having a chain-shaped alkyl group as a hydrophobic group (i.e., wherein R⁷ in formula (5) is “a straight-chain or branched alkyl group having from 12 to 22 carbons”) will allow the effect of achieving a rinse viscosity within a suitable range to be obtained. The use of a monomer having a ring structure as a hydrophobic group (i.e., wherein R⁷ in formula (5) is a “cyclohexyl group optionally having a substituent or a phenyl group optionally having a substituent”) will allow the effect of good texture when the copolymer is adsorbed to the hair to be obtained.

CH₂═C(R⁶)—COO—R⁷ (5)

(in formula (5), R⁶ is a hydrogen atom or a methyl group, R⁷ is a straight chain or branched alkyl group having from 12 to 22 carbons, a cyclohexyl group optionally having a substituent, or a phenyl group optionally having a substituent).

In formula (5), R⁶ preferably represents a methyl group. R⁷ is a straight-chain or branched alkyl group preferably having from 12 to 20 carbons, more preferably from 16 to 18 carbons; a cyclohexyl group optionally having a substituent; or a phenyl group optionally having a substituent. If the cyclohexyl group or phenyl group represented by R⁷ has a substituent, the substituent is preferably a straight-chain or branched alkyl group having from 1 to 4 carbons, more preferably a straight-chain or branched alkyl group having from 1 to 3 carbons. R⁷ is preferably a cyclohexyl group or a phenyl group not having a substituent, and is most preferably a cyclohexyl group.

One type of constituent unit derived from monomer (d) may be used in isolation in the copolymer (α) and copolymer (β) used in the present invention, or two or more types may be used in combination.

Examples of the (meth)acrylate monomer having an alkyl group having from 12 to 22 carbons represented by R⁷ in the formula (5) for monomer (d) above include myristyl (meth)acrylate, isostearyl (meth)acrylate, stearyl (meth)acrylate, behenyl (meth)acrylate, cetyl (meth)acrylate, lauryl (meth)acrylate, synthetic lauryl (meth)acrylate, (“synthetic lauryl (meth)acrylate” referring to an alkyl (meth)acrylate having an alkyl group with 12 carbon atoms and an alkyl group with 13 carbon atoms), and the like. Of these, (meth)acrylate monomers having an alkyl group with from 12 to 20 carbons are preferable as R⁷, and (meth)acrylate monomers having an alkyl group with from 16 to 18 carbons are more preferable. Examples of (meth)acrylate monomers in which R⁷ is a cyclohexyl group optionally having a substituent or a phenyl group optionally having a substituent include cyclohexyl (meth)acrylate, phenyl (meth)acrylate, and the like.

The proportion of constituent unit derived from monomer (d) in the copolymer (α) and copolymer (β) used in the present invention is preferably from 1 to 50 mass % with respect to the total masses of the copolymer (α) and the copolymer (β). Having the proportion of constituent unit derived from monomer (d) be at least 1 mass % allows effects of achieving a rinse viscosity within a sufficient range and satisfactory texture when the copolymer is adsorbed to the hair to be obtained, and having said proportion be no more than 50 mass % ensures the proportion of the constituent unit derived from the vinyl monomer (A) in the copolymer (α) and the copolymer (β), allowing conditioning ingredient adsorption to be increased. From considerations of enhancing these effects, the proportion of the constituent unit derived from monomer id) in copolymer (α) and copolymer (β) is more preferably at least 3 mass %, still more preferably at least 5 mass %, and especially more preferably at least 10 mass %; and more preferably no more than 40 mass %, especially preferably no more than 30 mass %.

<Constituent Unit Derived from Monomer (e)>

The copolymer (α) used in the cosmetic composition according to the First aspect of the present invention can also comprise the monomer (e) represented by formula (6) below as a constituent unit. The copolymer (β) used in the cosmetic composition according to the second aspect of the present invention preferably comprises the monomer (e) represented by formula (6) below as a constituent unit. The monomer (e) is capable of modifying the hydrophilicity and hydrophobicity of the copolymers (α) and (β), allowing for a smoothness of finger combing after drying to be obtained.

CH₂═C(R⁸)—CO—N(R⁹)R¹⁰ (6)

(in formula (6), R⁸ is a hydrogen atom or a methyl group, R⁹ and R¹⁰ are each independently a hydrogen atom or a straight chain or branched alkyl group having from 1 to 4 carbons).

In formula (6), R⁸ preferably represents a hydrogen atom. The sum of the number of carbons in R⁹ and R¹⁰ is preferably at least 2 and no more than 4. Examples of the monomer (e) include N,N-dimethyl (meth)acrylamide, N,N-diethyl (meth)acrylamide, and N-isopropyl (meth)acrylamide. Of these, monomers in which the sum of the number of carbons in R⁹ and R¹⁰ is 2 are preferable, with an example of such a monomer (e) being N,N-dimethyl (meth)acrylamide.

One type of constituent unit derived from monomer (e) may be used in isolation in the copolymer (α) and copolymer (β) used in the present invention, or two or more types may be used in combination.

From considerations of enhancing the aforementioned effects, the proportion of constituent unit derived from monomer (e) in the copolymer (α) and copolymer (β) used in the present invention is preferably at least 1 mass % with respect to the total masses of the copolymer (α) and the copolymer (β), more preferably at least 3 mass %, and still more preferably 5 mass %. Meanwhile, in order to ensure the proportion of constituent unit derived from the vinyl monomer (A) in the copolymer (α) and the copolymer (β) and increase the adsorption of the conditioning ingredients, the proportion is preferably no more than 50 mass %, more preferably no more than 30 mass %, still more preferably no more than 15 mass %, and especially preferably no more than 10 mass %.

<Constituent Units Derived from Other Monomers>

Apart from the monomers (a), (b), (c), (d), and (e) described above, the copolymer (α) and copolymer (β) used in the present invention may further comprise structural units derived from other vinyl monomers to the extent that the effects of the present invention are not lost. Examples of other vinyl monomers include nonionic monomers, amphoteric monomers, semipolar monomers, cationic monomers, as well as monomers containing a polysiloxane group. However, these other monomers do not include the aforementioned vinyl monomers (a), (b), (c), (d), and (e).

The amount of constituent units derived from other monomers comprised by the copolymer (α) and copolymer (β) used in the present invention can be determined, as appropriate, within a range such that the spirit of the present invention is not exceeded. For example, the amount can be appropriately determined within a range such that such that the affinity of the copolymer (α) and the copolymer (β) to the skin and hair is not hindered, or such that conditioning effects and the like are not hindered when the composition is used as a hair cosmetic; normally, the amount will be 40 mass % or less of the total mass of the copolymer, preferably 30 mass % or less, more preferably 20 mass % or less, and even more preferably 10 mass % or less.

However, because of the possibility that the formation of a complex with the cationic surfactant may be hindered if a cationic functional group is present in the copolymer (α) and copolymer (β) used in the present invention, the amount of cationic functional groups in the copolymer (α) and copolymer (β) used in the present invention is preferably low; for example, cationic functional groups preferably constitute no more than 10 mole % of all functional groups in the copolymer, and an essential lack of cationic functional groups is more preferable.

Examples of other monomers that can be comprised in the copolymer (α) and copolymer (β) used in the present invention are listed below, but the copolymer (α) and copolymer (β) used in the present invention may also comprise constituent units derived from monomers other than those listed below. Furthermore, it is acceptable to have only one type of component unit derived from other monomers in the copolymer used in the present invention, or to have two or more types of component units derived from the same or different types of other monomers.

Examples of nonionic monomers include esters of an alcohol and (meth)acrylic acid having from 1 to 11 carbons, amides of an alkylamine and (meth)acrylic acid having from 1 to 22 carbons, (meth)acryloyl morpholine, and the like.

Examples of amphoteric monomers include (meth)acryl esters having a betaine group, (meth)acrylamides having a betaine group and the like.

Examples of semipolar monomers include (meth)acrylate esters having an amine oxide group, (meth)acrylamides having an amine oxide group, and the like.

Examples of cationic monomers include (meth)acrylate esters having a quaternary ammonium group, (meth)acrylamides having a quaternary ammonium group, and the like.

Monomers containing a polysiloxane group are compounds that have a polysiloxane structure and a structure capable of covalently bonding with the copolymer (α) and the copolymer (β). These component units have high affinity to the silicone oils normally included in cosmetic compositions, and are thought to act by bonding the silicone oil to the other component units in the copolymer (α) and the copolymer (β), thus increasing the adsorption the of silicone oils to the skin and hair, particularly damaged hair. However, if a monomer containing a polysiloxane group is used as a copolymerization component, the amount of constituent units derived from a monomer containing a polysiloxane group comprised by the copolymer (α) and the copolymer (β) is no more than 40 mass %, preferably no more than 30 mass %, and still more preferably no more than 20 mass % so as not to impede the respective effects of the copolymer (α) and copolymer (β) used in the present invention.

A polysiloxane structure is a structure in which two or more repeating structural units expressed by the following formula (7) are linked.

—(SiR¹¹R¹²—O)— (7)

(in formula (7), R¹¹ and R¹² each represent an independent alkyl group having from 1 to 3 carbons, or a phenyl group)

Examples of structures capable of covalently bonding to the copolymer (α) and the copolymer (β) include structures having a vinyl structure, such as a (meth)acrylate ester or (meth)acrylamide, and capable of copolymerization with another monomer; structures having a functional group, such as a thiol, and capable of linking to the copolymer by chain transfer during polymerization; and structures having an isocyanate group, carboxylic acid group, hydroxyl group, amino group, or the like, and capable of reacting with and bonding to the functional groups of the copolymer; but the present invention is not limited thereto.

A plurality of these linkable structures can be present in one monomer containing a polysiloxane group. In the copolymer (α) and the copolymer (β), the polysiloxane structure can link by a graft structure to the main chain; or, conversely, the polysiloxane structure may be the main chain, to which the other structure is linked by a graft structure; or the polysiloxane structure and the other structure can be linked in a straight chain via a block structure.

The monomer containing a polysiloxane group is preferably expressed by the following formula (8).

CH₂═C(R¹³)-Q-(SiR¹⁴R¹⁵—O)ₛ—R¹⁶ (8)

(in formula (8), R¹³ represents a hydrogen atom or a methyl group, R¹⁴ and R¹⁵ independently represent an alkyl group having from 1 to 3 carbons or a phenyl group. R¹⁶ represents an alkyl group having from 1 to 8 carbons, Q represents a bivalent linking group or a direct bond, and s represents an integer from 2 to 200)

s is more preferably 3 or higher, and even more preferably 5 or higher. As s becomes larger, the affinity to silicone oils increases. Additionally, s is more preferably 50 or less. As s becomes smaller, copolymerization ability with other monomers is enhanced.

Q represents a bivalent linking group or a direct bond, but a linking group containing one or a combination of two or more of the structures suggested below is preferable. There is no particular limitation upon the number of structures in the combination, but the number will normally be 5 or less. Furthermore, the direction of the following structures are arbitrary (the polysiloxane group side can be on either end). In the following formulas, R represents an alkylene group having from 1 to 6 carbons, or a phenylene group.

—COO—R—

—CONH—R—

—O—R—R—

—R—

There is no particular limitation upon the monomer containing a polysiloxane group represented by the aforementioned formula (8) so long as it is encompassed by the formula; examples thereof include α-(vinyl phenyl) polydimethyl siloxane, α-(vinyl benzyloxy propyl) polydimethyl siloxane, α-(vinyl benzyl) polymethyl phenyl siloxane, α-(methacryloyl oxypropyl) polydimethyl siloxane, α-(methacryloyloxy propyl) polymethyl phenyl siloxane, α-(methacryloyl amino propyl) polydimethyl siloxane, and the like. One type of monomer containing a polysiloxane group can be used in isolation, or two or more types can be used in combination.

The monomer containing a polysiloxane group can be a commercial product. For example, α-(methacryloyloxy propyl) polydimethyl siloxane can be procured as Silaplane (Chisso Corporation); other examples include Silaplane FM0711 (molecular weight 1,000), Silaplane FM0721 (molecular weight 5.000), Silaplane FM0722 (molecular weight 10,000), and the like.

A crosslinking agent such as a multifunctional acrylate can be incorporated in the copolymer (α) and copolymer (β) used in the present invention in order to adjust the respective molecular weights or viscosities of the copolymer (α) and the copolymer (β), but it is preferably not to include a crosslinking agent, as will be discussed below.

### <Structural Analysis>

The amount of constituent units derived from the vinyl monomers (a), (b), (c), (d), and (e) and other monomers comprised in the copolymer (α) and the copolymer (β) can be measured using IR absorption or Raman scattering of carbonyl groups, amide bonds, polysiloxane structures, various types of functional groups, carbon backbone, or the like; ¹H-NMR of methyl groups or amide bond sites in the polydimethyl siloxane, as well as methyl groups or methylene groups adjacent thereto; or various types of NMR typified by ¹³C-NMR or the like.

### <Weight Average Molecular Weight>

The weight average molecular weight of the copolymer (α) and copolymer (β) used in the present invention is from 3,000 to 400,000. Having the weighted average molecular weight of the copolymer (α) and copolymer (β) used in the present invention be 3,000 or higher further enhances conditioning effects thanks to the adhesion of the complex with the cationic surfactant to the hair and skin. From considerations of further enhancing these effects, the weight average molecular weight of the copolymer (α) and copolymer (β) used in the present invention is preferably at least 5,000, more preferably at least 10,000. Meanwhile, having the weight average molecular weight of the copolymer (α) and copolymer (β) used in the present invention be no more than 400,000 allows for further improvement of texture after drying. From considerations of further enhancing these effects, the weight average molecular weight of the copolymer (α) and copolymer (β) used in the present invention is preferably no more than 200,000, more preferably no more than 100,000, still more preferably no more than 50,000, and most preferably no more than 30,000.

The molecular weight of the copolymer (α) and the copolymer (β) can be adjusted by controlling, for example, the degree of polymerization of the copolymers. Furthermore, the molecular weight can be controlled by increasing or decreasing the amount of crosslinking agent, such as polyfunctional acrylate or the like, that is added. However, if an even slightly excessive amount of crosslinking agent is added, molecular weight and viscosity will rapidly increase, leading to difficulties in control during industrial production. Therefore, it is most preferable not to add a crosslinking agent.

The weight average molecular weight of the copolymer (α) and the copolymer (β) can be measured by gel permeation chromatography (GPC). There is no particular limitation upon the developing solvent used in the gel permeation chromatography as long as it is a generally used solvent; for example, measurement can be performed using a water/methanol/acetic acid/sodium acetate blended solvent described below, using values taking polyethylene glycol or polystyrene as a reference substance.

### <Viscosity>

20 mass % ethanol solutions of the copolymer (α) and copolymer (β) used in the present invention preferably have viscosities of at least 5 mPa·s and no more than 20,000 mPa·s at 25° C. Viscosity is more preferably at least 10 mPa·s, even more preferably at least 15 mPa·s; and more preferably no more than 10,000 mPa·s, and even more preferably no more than 5,000 mPa·s. The viscosities of ethanol solutions of the copolymers are preferably at least 5 mPa·s and no more than 20,000 mPa·s from considerations of handling. Viscosity can be measured using a B-type viscometer or the like.

The copolymer (α) and copolymer (β) used in the present invention are preferably water soluble, and 30 mass % aqueous solutions thereof preferably have viscosities of at least 5 and no more than 20,000 mPa·s at 25° C. The viscosity is more preferably at least 10 even more preferably at least 15 mPa·s; and more preferably no more than 10,000 mPa·s, and even more preferably no more than 5.000 mPa·s. The viscosities of aqueous solutions of the copolymers are preferably at least 5 mPa·s and no more than 20,000 mPa·s from considerations of handling. Viscosity can be measured using a B-type viscometer or the like.

As in the case of weight average molecular weight, the viscosities of the copolymer (α) and the copolymer (β) can be adjusted by controlling, for example, the degree of polymerization of the copolymers, or by controlling by increasing or decreasing the amount of crosslinking agents, such as polyfunctional acrylate or the like, that is added.

### <Method of Producing the Copolymer (α) and the Copolymer (β)>

The copolymer (α) and copolymer (β) used in the present invention can be manufactured by blending monomers that provide the various component units or precursors thereof; copolymerizing by a method such as solution polymerization, suspension polymerization, emulsion polymerization, or the like; then adding polysiloxane structures, performing a condensation reaction, or the like as necessary.

Furthermore, the counter ions of the vinyl monomer (A) having a carboxyl group can be partially or completely replaced by a moiety other than a hydrogen ion by a neutralization reaction prior to polymerization, or partially replaced by a moiety other than a hydrogen ion by a neutralization reaction after polymerization and other reactions. These can be performed by appropriately selecting-based on the ease of synthesis.

The polymerization reaction is preferably performed in a hydrophilic solvent. Examples of hydrophilic solvents include ketone-based solvents such as acetone, methyl ethyl ketone, methyl isobutyl ketone, and the like; alcohol-based solvents such as methanol, ethanol, n-propanol, i-propanol, n-butanol, i-butanol, sec-butanol, and the like; water, and the like. These solvents can be used individually, or two or more types can be used in combination. Of these, alcohol-based solvents are preferably used.

Non-limiting examples of employable polymerization initiators include azo compounds such as 2,2′-azobis isobutyronitrile, 2,2′-azo bis(2,4-dimethyl valeronitrile), 2,2′-azo bis(4-methoxy-2,4-dimethyl valeronitrile), dimethyl-2,2′-azo bisisobutyrate, 2,2′-azo bis(2-methyl butyronitrile), 1,1′-azo bis(l-cyclohexane carbonitrile), 2,2′-azo bis(2-methyl-N-(2-hydroxyethyl)-propionamide), 2,2′-azo bis(2-amidinopropane)dihydrochloride, and the like; peroxides such as benzoyl peroxide, dicumyl peroxide, di-t-butyl peroxide, lauroyl peroxide and the like; persulfates; and redox systems thereof. The amount of polymerization initiator is preferably in a range from 0.01 to 5 mass % with respect to the total amount of monomer.

The polymerization reaction is normally performed for from 1 to 30 hours in an inert gas environment such as nitrogen, argon or the like, preferably at a temperature of from 30 to 120° C., more preferably at from 40 to 100° C. After polymerization is complete, the produced copolymers can be segregated from the reaction solution by an appropriate means, such as distilling away the solvent or adding a poor solvent. This copolymer can be used as-is or be further refined, and used, for example, to produce a cosmetic material. Refining can be performed by, for example, reprecipitation, solvent washing, membrane separation, or an appropriate combination of such methods as necessary,

### <Neutralizing the Carboxyl Groups>

Part of the total amount of carboxyl groups belonging to the copolymer (α) used in the present invention may be neutralized. Neutralizing the carboxyl groups increases the ionicity thereof, enhancing interaction with the cationic surfactant. On the other hand, too high a level of ionicity will break down the structure of the lamellar gel formed by the water, the cationic surfactant, and the higher alcohol, so it is preferable that only few carboxyl groups be neutralized.

For the reasons given above, it is preferable that from 50 to 100 mol % of the carboxyl groups belonging to the copolymer (α) used in the cosmetic composition according to the first aspect of the present invention be added in an un-neutralized state. The proportion of unneutralized carboxyl groups is more preferably at least 60 mol %, still more preferably at least 80 mol %, and especially preferably at least 90 mol %. It is most preferable for all of the carboxyl groups to be added in an tin-neutralized state.

Meanwhile, from 50 to 100 mol % of the carboxyl groups belonging to the copolymer (β) used in the cosmetic composition according to the second aspect of the present invention are added in an un-neutralized state. The proportion of un-neutralized carboxyl groups in the copolymer (β) is preferably at least 60 mol %, more preferably at least 80 mol %, and still more preferably at least 90 mol %. It is most preferable for all of the carboxyl groups to be added in an un-neutralized state.

In the cosmetic compositions according to the first aspect and the second aspect of the present invention, if neutralization is not deliberately performed upon the copolymer (α) or the copolymer (β) prior to being added, the carboxyl groups within the copolymers will be considered un-neutralized if the pH after the copolymers have been added is 6.9 or less, more preferably 6.5 or less, even if alkaline ingredients are present among the ingredients contained in the cosmetic composition according to the first aspect of the present invention.

Examples of neutralizing agents that can be used with the copolymer (α) and the copolymer (β) include alkali metal hydroxides such as sodium hydroxide and potassium hydroxide; alkali earth metal hydroxides such as calcium hydroxide and magnesium hydroxide; ammonia; and amines such as diethylamine, triethylamine, diethanolamine, and 2-amino-2-methyl propanol.

### [Formulation of the Cosmetic Composition]

The cosmetic composition according to the first aspect of the present invention comprises from 0.05 to 5 mass % of the abovementioned copolymer (α) used in the present invention and from 55 to 99.6 mass % water, and is preferably prepared with a cationic surfactant, a higher alcohol, and a silicone oil further included in its formula. The cosmetic composition according to the second aspect of the present invention comprises from 0.05 to 5 mass % of the abovementioned copolymer (β) used in the present invention and from 55 to 99.6 mass % water, and is preferably prepared with a cationic surfactant, a higher alcohol, and a silicone oil further included in its formula. Unless otherwise specified, the following description of the formulation of the cosmetic composition pertains to both the cosmetic composition according to the first aspect and the cosmetic composition according to the second aspect of the present invention.

The water, cationic surfactant, and higher alcohol or the like included as necessary in the cosmetic composition according to the present invention form what is called a lamellar gel structure when heated and mixed, allowing smoothness and a moisturized feel to be imparted, and superior conditioning effects can be obtained by adding the copolymer (α) or copolymer (β) used in the present invention thereto. Furthermore, a silky feel can be imparted by adding a silicone oil to the cosmetic composition of the present invention.

A “cosmetic composition using the present invention” refers to a composition for any cosmetic material used on the hair and skin, examples including a wide variety of compositions for various cosmetic materials such as shampoos, rinses, conditioners, treatments, hair colorants, permanent wave agents, out bath treatments, hair packs, hairsprays, hair foams, styling agents, body shampoos, makeup cleansers, hand soaps, emulsions, toilet waters, lotions, creams, beauty scrums, sunscreens, foundations, lipsticks, mascaras, eyeshadows, depilation agents, and the like. All modes of use are encompassed, such as applying the product to the skin or hair, working in, and washing (rinsing) out, or not rinsing out.

### <Copolymer (α) and Copolymer (β)>

The inclusion of at least 0.05 mass %, with respect to the total amount of cosmetic composition, of the copolymer (α) or the copolymer (β) used in the present invention within the cosmetic composition according to the present invention increases adsorption to the skin or hair, and imparts conditioning effects such as smoothness during rinsing and a silky feel after drying.

From considerations of heightening these effects, the cosmetic composition according to the first aspect of the present invention more preferably comprises at least 0.1 mass %, still more preferably at least 0.2 mass %, of the copolymer (α) used in the present invention. However, in order to eliminate coarse/rough feeling after drying and increase moisturization, the amount of the copolymer (α) used in the present invention included in the cosmetic composition according to the first aspect of the present invention is no more than 5 mass %, preferably no more than 3 mass %. Similarly, the cosmetic composition according to the second aspect of the present invention more preferably comprises at least 0.1 mass % of the copolymer (β) used in the present invention, still more preferably at least 0.2 mass %. However, in order to eliminate coarse/rough feeling after drying and increase moisturization, the amount of the copolymer (β) used in the present invention included in the cosmetic composition according to the first aspect of the present invention is no more than 5 mass %, preferably no more than 3 mass %.

In the cosmetic composition according to the present invention, one type each of the copolymer (α) and copolymer (β) used in the present invention may be used, or two or more types thereof may be used.

### <Solvent or Dispersion Medium>

The copolymer (α) and copolymer (β) used in the present invention may be dissolved or dispersed in a solvent or dispersion medium capable of being used with the cosmetic composition. Examples of solvents and dispersing media include water and lower alcohols such as ethanol, isopropanol, butanol, and the like, but the cosmetic composition according to the present invention at least contains water.

The water content of the cosmetic composition according to the present invention is at least 55 mass % with respect to the total mass of the cosmetic composition according to the present invention. Including at least 55 mass % water keeps viscosity from becoming too great and allows the composition to be kept at a manageable viscosity, improving the feel of the product during application. The water content is more preferably at least 60 mass %. However, the water content is preferably no more than 99.6 mass %.

A water content within the range described above allows the cosmetic composition according to the present invention to exhibit a suitable degree of viscosity, and improves smoothness during application and adsorption to the skin or hair.

### <Cationic Surfactant>

The cosmetic composition according to the present invention preferably comprises a cationic surfactant.

There is no particular limitation upon the cationic surfactant included in the cosmetic composition according to the present invention; examples include alkyl trimethyl ammonium salts such as stearyl trimethyl ammonium chloride and lauryl trimethyl ammonium chloride; alkyl pyridinium salts such as cetylpyridinium chloride; dialkyl dimethyl ammonium salts such as distearyl dimethyl ammonium chloride; poly(N,N′-dimethyl-3,5-methylene piperidinium); alkyl quaternary ammonium salts; alkyl dimethyl benzyl ammonium salts; alkyl isoquinoline salts; dialkyl morphonium salts; polyoxyethylene alkyl amines; alkyl amine salts; polyamine fatty acid derivatives; amyl alcohol fatty acid derivatives; benzalkonium chloride; benzethonium chloride; and the like.

Cationic surfactants may be used singly or in combinations of two or more.

These cationic surfactants are preferably comprised in an amount of no more than 20 with respect to the total mass of the cosmetic composition according to the present invention, more preferably no more than 10 mass %. Meanwhile, in order to sufficiently impart the effects of the cationic surfactant, the content thereof is preferably at least 0.1 mass % with respect to the total mass of the cosmetic composition, more preferably at least 1 mass %.

### <Higher Alcohols>

The cosmetic composition according to the present invention preferably comprises a higher alcohol. In the context of this specification, “higher alcohol” refers to an alcohol having from 12 to 24, preferably a monovalent alcohol. Specific examples of higher alcohols include cetyl alcohol, stearyl alcohol, and the like.

Using a higher alcohol along with water and a cationic surfactant forms what is called a lamellar gel structure, which is capable of imparting the cosmetic composition with smoothness and moisturizing effects. For this reason, a higher alcohol is preferably used along with water and the cationic surfactant. Higher alcohols may be used singly or in combinations of two types or more, but, in order to keep the viscosity of the cosmetic composition at a suitable level and improve smoothness during application, the total higher alcohol content with respect to the total mass of the cosmetic composition is preferably no more than 20 mass %, more preferably no more than 10 mass %. Meanwhile, in order to sufficiently impart the effects of the higher alcohol, the content thereof is preferably at least 0.1 mass % with respect to the total mass of the cosmetic composition, more preferably at least 1 mass %.

### <Silicone Oils>

The cosmetic composition according to the present invention preferably comprises a silicone oil. There is no particular limitation upon the type of silicone oil used in the cosmetic composition according to the present invention; examples include polydimethyl siloxane, polydimethyl siloxane alkylene oxide copolymer, amino-modified polydimethyl siloxane, polyether-modified polydimethyl siloxane, methylphenyl polysiloxane, epoxy-modified polydimethyl siloxane, fluorine-modified polydimethyl siloxane, alcohol-modified polydimethyl siloxane, alkyl-modified polydimethyl siloxane, alkoxy-modified polydimethyl siloxane, cyclic silicone, and the like. These silicone oils are disclosed in Unexamined Japanese Patent Application Publication 2000-336018.

Examples of commercially available polydimethyl siloxanes include “KF-96H-1,000,000” (available from Shin-etsu Chemical Co., Ltd.); “SH200”, “BY11-007”, and “BY22-029” (all available from Dow Corning Toray Silicone Co., Ltd.); “TSF451” (available from Toshiba Silicone Co., Ltd.); and “L-45” (available from Nippon Unicar Co., Ltd.).

An example of a commercially available polydimethyl siloxane alkylene oxide copolymer is “F-178-21” (available from Nippon Unicar Co., Ltd.).

Examples of amino-modified polydimethyl siloxanes include those having an amino alkyl group such as an aminoethyl group, an aminopropyl group, or the like; and amino groups to which a propylene glycol has been added (amino glycol-modified). Siloxanes having a substituent such as an alkyl group or a hydroxy 1 group are also acceptable. An alkyl group is preferably a straight-chain or branched alkyl group having from 1 to 12 carbons.

These amino-modified polydimethyl siloxanes can be obtained by copolymerizing a dimethyl siloxane and a siloxane having the aforementioned aminoalkyl group, or by subsequent modification of a polydimethyl siloxane by the compound having the aforementioned aminoalkyl group to introduce an aminoalkyl group into the skeleton of the polydimethyl siloxane. Examples of commercially available amino-modified polydimethyl siloxanes include “US AR SILICONE ALE56” (available from Union Carbide); “ABIL9905” (available from Goldschmidt); “KF857”, “KF867” and “KF865” (all available from Shin-etsu Chemical Co., Ltd.); “SM8702C” and “JP8500” (available from Dow Corning Toray Silicone Co. Ltd.); “FZ-3707” (Nippon Unicar Co., Ltd.); and the like.

Examples of polyether-modified polydimethyl siloxanes include those having a monomeric skeleton such as oxyethylene, oxypropylene, oxybutylene, oxypentamethylene, oxyhexamethylene, or oxy(2,2-dimethyl)propylene; those having a single copolymer frame such as polyoxyethylene, polyoxypropylene, polyoxybutylene, polyoxypentamethylene, or polyoxyhexamethylene; or those having an oxyalkylene group with a copolymer skeleton such as poly(oxyethylene oxypropylene) copolymer. These may have a substituent such as a hydroxyl group or an alkyl group. An alkyl group is preferably a straight-chain or branched alkyl group having from 1 to 12 carbons. A poly ether-modified polydimethyl siloxane having an oxyalkylene group in the copolymer skeleton is especially preferable.

Methods of producing such modified polydimethyl siloxanes having oxyalkylene groups include copolymerizing a dimethyl siloxane and a siloxane having an oxyalkylene group; or subsequently modifying a polydimethyl siloxane with a compound having the aforementioned oxyalkylene group to introduce an oxyalkylene group into the polydimethyl siloxane skeleton.

A most preferable polyether-modified polydimethyl siloxane is one obtained by copolymerizing a dimethyl siloxane and a siloxane having an oxyalkylene group in the copolymer skeleton.

Examples of commercially available polyether-modified polydimethyl siloxanes include “KF945A”, “KF351A”, and “KF354A” (all available from Shin-etsu Chemical Co., Ltd.); “SH3771C” and “SH3749” (available from Dow Corning Toray Silicone Co., Ltd.); “L-7602C” and “L-720” (available from Nippon Unicar Co., Ltd.); and “SF1066” (available from GE).

Examples of commercially available methylphenyl polysiloxanes include “KF56” (available from Shin-etsu Chemical Co., Ltd.), “SH5” (available from Shin-etsu Chemical Co., Ltd.), “PS922” (available from Chisso Corp.) and “L-930” (available from Nippon Unicar Co., Ltd.).

Examples of commercially available fluorine-modified polydimethyl siloxanes include “X-22-820” (Shin-etsu Chemical Co., Ltd.) and “PS182” (available from Chisso Corp.).

Examples of commercially available alcohol-modified polydimethyl siloxanes include “KF851” (Shin-etsu Chemical Co., Ltd.); “FM4411” (Chisso Corp.); and “FZ-3722” and “F-235-21” (available from Nippon Unicar Co., Ltd.).

Examples of commercially available alkyl-modified polydimethyl siloxanes include “KF410” and “KF-413” (available from Shin-etsu Chemical Co., Ltd.); and “PS130” and “PS137” (available from Chisso Corp.).

Examples of alkoxy-modified polydimethyl siloxanes include “PS912” (available from Chisso Corp.) and “FZ-3701” (available from Nippon Unicar Co., Ltd.).

Examples of cyclic silicones include “SFI244”, “SH245”, and “SH246” (available from Dow Corning Toray Silicone Co., Ltd.).

One type of silicone oil may be used in isolation, or two or more types may be used in combination. Because excessive amounts of silicone oil will lead to a heavy texture after application to the hair and drying, the amount of silicone oil is preferably no more than a total of 10 mass % with respect to the total mass of the cosmetic composition, more preferably no more than 8 mass %. Meanwhile, in order to sufficiently impart the effects of the silicone oil, the content thereof is preferably at least 0.1 mass % with respect to the total mass of the cosmetic composition, more preferably at least 1 mass %.

Meanwhile, the silicone oil contents of the cosmetic composition according to the first aspect of the present invention and the cosmetic composition according to the second aspect of the present invention are preferably less than 0.1 mass %. A silicone oil content of less than 0.1 allows a cosmetic composition having a superior smoothness of finger combing after drying to be obtained. From this perspective, it is preferable not to include any silicone oil.

### <Other Ingredient>

Along with the copolymer (α) or copolymer (β), water, cationic surfactant, higher alcohol, and silicone oil used in the present invention, hydrocarbon oils, moisturizing agents (water-soluble polymers); cationic polymers, anionic polymers, nonionic polymers, amphoteric polymers, and other polymers; various surfactants apart from the cationic surfactant; pH adjusting agents; preservatives; thickening agents; and the like can also be used in combination, as appropriate, to the extent that the effects of the cosmetic composition according to the present invention.

Examples of hydrocarbon oils include liquid paraffins such as isoparaffin, solid paraffin, petrolatum, ceresin, microcrystalline wax, and the like. These hydrocarbon oils may be used singly or in combinations of two types or more, but preferably constitute no more than 5 mass % massed total with respect to the total mass of the cosmetic composition. One type of these hydrocarbon oils may be used alone, or a desired combination of a plurality of types at a desired ratio may be used.

Examples of moisturizing agents include glycerin, dipropyleneglycol, 1,3-butanediol, erythritol, and other polyhydroxy alcohols; and methylcellulose, hyaluronic acid, and other water-soluble polymers. Examples of cationic polymers include cation-modified cellulose ether derivatives, cation-modified galactomannan polysaccharides, polydimethyl diallyl ammonium halides, copolymers of dimethyl diallyl ammonium halides and acrylamides, and the like. Examples of anionic polymers and nonionic polymers include acrylic acid derivatives (hydroxyethyl acrylate/methoxyethyl acrylate copolymers, polyacrylamides, etc.), vinylpyrrolidone derivatives (polyvinylpyrrolidone, vinylpyrrolidone/vinyl acetate copolymers, etc.); and examples of amphoteric polymers include dimethyl diallyl ammonium derivatives (acrylamide/acrylic acid/dimethyl diallyl ammonium chloride copolymers, acrylic acid/dimethyl diallyl ammonium chloride copolymers, etc.). The cationic polymers, anionic polymers, nonionic polymers, and amphoteric polymers listed above are assumed not to be comprised in the copolymers used in the present invention. The abovementioned moisturizing agents can be used singly or in desired combinations of a plurality of types at desired ratios, but preferably constitute no more than 5 mass % total with respect to the total mass of the cosmetic composition.

Examples of surfactants other than cationic surfactants include anionic surfactants, hydrophilic nonionic surfactants, amphoteric surfactants, semipolar surfactants, and the like. Specific examples of surfactants other than cationic surfactants are given below, but, if used, these should preferably constitute no more than 5 mass % total with respect to the total mass of the cosmetic composition.

Examples of anionic surfactants include those generally used in typical cosmetic compositions, such as α-olefin sulfonates, higher alcohol sulfuric acid ester salts, polyoxyethylene alkyl ether sulfuric acid ester salts, paraffin sulfonates, polyoxyethylene alkyl ether carboxylic acid ester salts, alkyl sulfosuccinates, N-acyl-β-alanine salts, N-acyl glutamates, acyl methyl taurines, and the like. Examples of counterions for these anionic surfactant include sodium, potassium, ammonium, triethanolamine, diethanolamine, and the like. One type of these anionic surfactants may be used alone, or a desired combination of a plurality of types at a desired ratio may be used.

Examples of hydrophilic nonionic surfactants include polyoxyethylene (hereafter, “POE”) sorbitan fatty esters (for example, POE sorbitan monooleates, POE sorbitan monostearates, POE sorbitan monooleates, POE sorbitan tetraoleates, etc); POE sorbitol fatty esters (for example, POE sorbitol monolaurates, POE sorbitol monooleates, POE sorbitol pentaoleates, POE sorbitol monostearates, etc.); POE glycerin fatty esters (for example, POE glycerin monostearates, POE glycerin monoisostearates, POE glycerin triisoslearates, etc.); POE fatty esters (for example, POE distearates, POE monodioleates, ethylene glycol distearate, etc.); POE alkyl ethers (for example, POE lauryl ethers, POE oleyl ethers, POE stearyl ethers, POE behenyl ethers. POE 2-octyl dodedeycl ethers. POE cholestanol ethers, etc.); pluronics (for example. Pluronic, etc.); POE/polyoxypropylene (hereafter, “POP”) alkyl ethers (for example POE.POP cetyl ethers, POE.POP-2-decyl tetradecyl ethers. POE.POP monobutyl ethers, POE.POP hydrogenated lanolin, POE.POP glycerin ethers, etc.); tetraPOE.tetraPOP ethylene diamine condensates (for example, Tetronics, etc.); POE castor oil/hydrogenated castor oil derivatives (for example, POE castor oil. POE hydrogenated castor oil, POE hydrogenated castor oil monoisostearate, POE hydrogenated castor oil triisostearate, POE hydrogenated castor oil monopyroglutamate monoisostearate diesters, POE hydrogenated castor oil maleate, etc.); POE beeswax/lanolin derivatives (for example, POE sorbitol beeswax, etc.); alkanolamides (for example, palm oil fatty acid diethanolamides, monoethanol amide laurate, fatty acid isopropanolamides, etc.); POE propyleneglycol fatty esters; POE alkylamines; POE fatty amides; sucrose fatty esters; alkyl ethoxydimethylamine oxides; and trioleyl phosphate. One type of these hydrophilic nonionic surfactants may be used alone, or a desired combination of a plurality of types at a desired ratio may be used.

Examples of amphoteric surfactants include 2-undecyl-N,N,N-(hydroxyethyl carboxymethyl)-2-imidazoline sodium, 2-cocoyl-2-imidazolinium hydroxide-1-carboxy ethyloxy-2nitrate, and other imidazoline-based amphoteric surfactants; and 2-heptadecyl-N-carboxymethyl-N-hydroxyethyl imidazolinium betaine, betaine lauryl dimethylaminoacetate, alkyl betaine, amido betaine, sulfobetaine, and other betaine-based surfactants. One type of these amphoteric surfactants may be used alone, or a desired combination of a plurality of types at a desired ratio may be used.

Examples of semipolar surfactants include lauramine oxides (lauryl dimethylamine oxides), and can be preferably used. One type of these semipolar surfactants may be used alone, or a desired combination of a plurality of types at a desired ratio may be used.

Such surfactants are generally commercially available, and can be used as-is.

Examples of pH adjusting agents include citric acid and tartaric acid. If a pH adjusting agent is used, the amount thereof preferably constitutes no more than 0.1 mass % with respect to the total mass of the cosmetic composition. One type of these pH adjusting agents may be used alone, or a desired combination of a plurality of types at a desired ratio may be used.

An example of a preservative is sodium benzoate. If a preservative is used, the amount thereof preferably constitutes no more than 0.1 mass % with respect to the total mass of the cosmetic composition. One type of preservative may be used alone, or a desired combination of a plurality of types at a desired ratio may be used.

Examples of thickening agents include hydroxymethylcellulose, hydroxyethylcellulose, and the like. If a thickening agent is used, the amount thereof preferably constitutes no more than 5 mass % with respect to the total mass of the cosmetic composition. One type of these thickening agents may be used alone, or a desired combination of a plurality of types at a desired ratio may be used.

Apart from the ingredients listed above, various animal- and plant-derived natural extracts and derivatives thereof, organic acids such as lactic acid, inorganic salts such as sodium chloride, amino acids (glutamic acid and salts thereof, arginine and salts thereof, glycine, etc.), antioxidants. UV absorber, bactericides, chelating agents, perfume, colors, metal sequestering agents (edetic acid salts, etc.), foam boosters, and the like may be included, as appropriate, to the extent that the effects of the present invention are not impeded. If used, such ingredients preferably constitute no more than 20 mass % total of the total mass of the cosmetic composition.

The cosmetic composition according to the present invention does not preclude the use of ingredients other than those listed above.

The viscosity of the cosmetic composition according to the present invention will vary according to application: for example, if the cosmetic composition according to the present invention is used as a hair cosmetic, the viscosity thereof at 25° C. is preferably at least 300 mPa·s and no more than 100.000 mPa·s. The viscosity is more preferably at least 1,000 mPa·s, still more preferably 2,000 mPa·s, especially preferably 5,000 mPa·s, and most preferably 10,000 and more preferably no more than 90,000 mPa·s, and still more preferably no more than 80,000 mPa·s. The viscosity of the cosmetic composition is preferably at least 300 mPa·s and no more than 100,000 mPa·s from considerations of handling. The viscosity of the cosmetic composition, as shown in the working examples listed in the present specification, can be measured using a B-type viscometer or a B8H-type viscometer.

### [Hair Cosmetic]

The cosmetic composition according to the present invention is especially useful as a hair cosmetic. In the present specification, there is no particular limitation upon the type of hair cosmetic; examples Include shampoos, rinses, conditioners, treatments, outbath treatments, hair dyes, perming agents, hair packs, hair sprays, hair foams, styling agents, and the like. Of these, the hair cosmetic according to the present Invention is especially useful for shampoos, rinses, conditioners, treatments, outbath treatments, and other conditioning agents from which conditioning effects are demanded.

Among the various types of hair cosmetics, the cosmetic composition according to the present invention is especially effective as a hair treatment cosmetic. The primary purpose of hair treatment cosmetics is to impart conditioning effects after washing the hair with shampoo or the tike, with there being both types that are rinsed out with water after application and types that are not rinsed out after application. Examples of cosmetics that are rinsed out with water after application include rinses, conditioners, treatments, and other so-called rinse-off conditioners; and examples of cosmetics that are not rinsed out include outbath treatments and so-called leave-on conditioners. Of these, the cosmetic composition according to the present invention is especially useful as a rinse-off conditioner,

### EXAMPLES

The present invention will now be described in greater detail through the use of working examples, but is in no way limited to these working examples as long as the scope of the present invention is not exceeded. The various production conditions and evaluation results values in the following working examples signify preferable upper-limit and lower-limit values in the embodiment of the present invention, and a preferable range can be a range defined by a combination of the aforementioned upper-limit and lower-limits values and the values for the following working examples, or by a combination of values for different working examples,

### [Copolymer Production]

### <Producing Copolymer (1)>

100 parts by mass ethanol was introduced into a reaction vessel equipped with a reflux condenser, a dropping funnel, a thermometer, a nitrogen gas injection tube, and a stirrer; a liquid monomer blend containing 40 parts by mass methoxy polyethylene glycol (2 mol) methacrylate, 60 parts by mass acrylic acid, and 100 parts by mass ethanol was added to the dropping funnel; and the reaction vessel was purged with nitrogen and heated to 80° C. 0.5 parts by mass dimethyl-2,2′-azo bisisobutyrate (V-601: produced by Wako Pure Chemical Industries) was added to the reaction vessel, and the liquid copolymer blend was added dropwise over 2 hours. Once dropwise addition was finished, the blend was reacted for 8 hours, after which distilled water was added as the ethanol solvent was distilled away to obtain an aqueous solution of copolymer 11).

### <Producing Copolymers (2) to (12)>

Copolymers (2) to (12) were produced in a manner similar to that of copolymer (1), except that the ratios shown in Table 1 of the monomers shown in Table 1 were used (although “copolymer (9)” is not a “copolymer” but rather a “polymer” using a single monomer, it has been labeled “copolymer (9)” for convenience).

### [Measuring Copolymer Physical Properties]

### <Measuring Weight Average Molecular Weight>

The weight average molecular weight of copolymers (1) to (8), (10), and (11) were determined using gel permeation chromatography (devices: “SC8010, SD8022, RI8020, CO8011, PS8010” manufactured by Tosoh Corporation; column: “Wakopak (Wakobeads G-50)” manufactured by Wako Pure Chemical Industries; developing solvent: water/methanol/acetic acid/sodium acetate=6/4/0.3/0.41), with polyethylene glycol as a reference substance.

The weight average molecular weight of copolymer (9) was determined via gel permeation chromatography (devices: “SC8020, SD8022, CCPM-II, RI8020, CO8020” manufactured by Tosoh Corporation; column: “G6000Hx1, G4000Hx1, G2500Hx1” manufactured by Tosoh Corporation; developing solvent: tetrahydrofuran) using polystyrene as a reference substance.

The results obtained are shown in Table 1,

TABLE 1
Types and proportions (parts by mass) of vinyl monomers used to produce polymers, and polymer weight average molecular weights

| | | | | | | | | | | | Copoly- | Copoly- | Copoly- |
|---|---|---|---|---|---|---|---|---|---|---|---|---|---|
| | | Copoly- | Copoly- | Copoly- | Copoly- | Copoly- | Copoly- | Copoly- | Copoly- | Copoly- | mer | mer | mer |
| | | mer (1) | mer (2) | mer (3) | mer (4) | mer (5) | mer (6) | mer (7) | mer (8) | mer (9) | (10) | (11) | (12) |
| Monomer | Acrylic acid | 60 | 80 | 60 | 60 | 50 | 50 | 80 | 80 | 100 | 80 | 30 | 65 |
| (a) | | | | | | | | | | | | | |
| Monomer | 2-methacryloyl oxyetyl | | | | | 50 | | | | | | | |
| (b) | hexahydrophthalate | | | | | | | | | | | | |
| | 2-acryoyl oxy | | | | | | 50 | | | | | | |
| | ethyl succinate | | | | | | | | | | | | |
| Monomer | Methoxy PEG | 40 | | | | | | | | | | | |
| (c) | (2 mol) | | | | | | | | | | | | |
| | Methacrylate* | | | | | | | | | | | | |
| | methoxy PEG | | 20 | | | | | | | | | | 35 |
| | (4 mol) | | | | | | | | | | | | |
| | methacrylate* | | | | | | | | | | | | |
| | Hydroxyethyl acrylate | | | 40 | | | | | | | | | |
| | Hydroxyethyl | | | | 40 | | | | | | | 70 | |
| | acrylamide | | | | | | | | | | | | |
| Monomer | Isostearyl methacrylate | | | | | | | 20 | | | | | |
| (d) | Cyclohexyl | | | | | | | | 20 | | | | |
| | methacrylate | | | | | | | | | | | | |
| Monomer | Dimethyl acrylamide | | | | | | | | | | 20 | | |
| (e) | | | | | | | | | | | | | |

| | | | | | | | | | | | | |
|---|---|---|---|---|---|---|---|---|---|---|---|---|
| Weight average molecular | 22,000 | 19,000 | 23,000 | 30,700 | 5,500 | 12,300 | 10,000 | 9,000 | 33,800 | 20,000 | 22,800 | 20,000 |
| weight | | | | | | | | | | | | |

In Table 1, blank fields indicate that the vinyl monomer was not used.
*Methoxy PEG (n mol)methacrylate is methoxy polyethylene glycol (n mol) methacrylate, and “n” indicates that r = n in formula (4).

### [Measuring and Evaluating Physical Properties of the Cosmetic Compositions]

The physical properties of the cosmetic compositions according to the following working examples and comparative examples were measured and evaluated as described below,

### <Damaged Hair>

A root-aligned bundle (untreated hair, 10 g×30 cm; manufactured by Beaulax) of 100% black human hair was used as an evaluation tuft, and a similar bundle subjected to bleaching was used for “damaged hair”. Specifically, a mixture of 12 g Milbon “Promatiz Flaeve Oxytane 6.0 (6% hydrogen peroxide cream) and 6 g Meros “Powder Bleach MR2” was applied as a bleaching agent to one of these bundles, the bundle was washed with water after being left to stand for 30 minutes, then washed with lauroyl (EO) 3 sodium sulfate (polyoxyethylene (3) lauryl ether sodium sulfate) to obtain damaged hair. A 5 g bundle of this damaged hair was used as a damaged hair sample bundle.

### <Evaluation of Smoothness During Application>

The damaged hair sample bundle was washed and rinsed using 25 mass % polyoxyethylene (3) lauryl ether sodium sulfate as a shampoo, after which the various cosmetic compositions described above were applied, and smoothness of finger combing when rinsed in 40° C. flowing water was evaluated according to the following four stages. The cosmetic composition prepared using copolymer (11) was used as a comparative standard product.

+2: Superior to comparative standard product

+1: Somewhat superior to comparative standard product

0: Comparable to comparative standard product

−1: Inferior to comparative standard product

<Evaluating Reduced Coarse/Rough Feeling of Hair after Drying>

The damaged hair sample bundle was washed and rinsed using 25 mass % polyoxyethylene (3) lauryl ether sodium sulfate as a shampoo, after which the various cosmetic compositions described above were applied, the hair bundle was rinsed in 40° C. flowing water, the hair bundle was naturally dried at 23° C. and 60% RH for one day, and the reduced coarse/rough feeling of the hair after drying was evaluated according to the following four stages. The evaluation of reduced coarse/rough feeling of hair was an evaluation of the degree to which the coarse/rough feeling of the damaged hair was reduced, and is a combined evaluation of uniformity of hair surface and hair bundle softness. The cosmetic composition prepared using copolymer (11) was used as a comparative standard product.

+2: Superior to comparative standard product

+ 1: Somewhat superior to comparative standard product

0: Comparable to comparative standard product

−1: Inferior to comparative standard product

<Evaluation of Smoothness of Finger Combing after Drying>

The damaged hair sample bundle was washed and rinsed using 25 mass % polyoxyethylene (3) lauryl ether sodium sulfate as a shampoo, after which the various cosmetic compositions described above were applied, the hair bundle was rinsed in 40° C. flowing water and patted dry with a towel, and the smoothness when combing by the fingers through the hair bundle was evaluated according to the following four stages. The cosmetic composition prepared using copolymer (11) was used as a comparative standard product.

+2: Superior to comparative standard product

+1: Somewhat superior to comparative standard product

0: Comparable to comparative standard product

−1: Inferior to comparative standard product

<Evaluation of Manageability after Drying>

The damaged hair sample bundle was washed and rinsed using 25 mass % polyoxyethylene (3) lauryl ether sodium sulfate as a shampoo, after which the various cosmetic compositions described above were applied, the hair bundle was rinsed in 40° C. flowing water and patted dry with a towel, and the tips of the hair were raised and dropped to evaluate hair manageability (reduced spreading of hair tips) according to the following four stages. The cosmetic composition prepared using copolymer (11) was used as a comparative standard product.

+2: Superior to comparative standard product

+1: Somewhat superior to comparative standard product

0: Comparable to comparative standard product

−1: Inferior to comparative standard product

### <Measuring Viscosity>

The viscosity of the prepared cosmetic composition was measured at 25° C. using the #4 of a B-type viscometer set to 12 rpm.

### <Measuring Silicone Oil Adsorption>

The damaged hair sample bundle described above was washed and rinsed using 25 polyoxyethylene (3) lauryl ether sodium sulfate as a shampoo, cosmetic compositions prepared according to the following working examples and comparative examples were applied, and the hair bundle was rinsed in 40° C. flowing water, after which the silicone oil adsorbed to the hair was extracted and polydimethyl siloxane adsorption was evaluated via NMR measurement (metered dose of H of Si—CH₃ using ¹H-NMR) of the extracted fluid.

Extraction was performed by submerging the hair bundle in a 4:1 chloroform/methanol solvent and performing ultrasonic treatment, vacuum drying the extracted solvent, the residue was dissolved in heavy chloroform, dimethyl terephthalate was added as an internal marker and the concentration thereof determined, and the amount of polydimethyl siloxane adsorption was indicated in terms of such concentration.

### Working Examples/Comparative Examples of Cosmetic Composition According to the First Aspect of the Present Invention

Working examples and comparative examples of the cosmetic composition according to the first aspect of the present invention will be described below.

### Preparation/Evaluation of Cosmetic Compositions

### Working examples 1-1 to 1-11, Comparative Example 1-1

Using copolymers (1) to (12), cosmetic compositions having the compositions shown in Table 2 were prepared according to a normal method. Masses for each ingredient are in terms of active ingredients, and the accompanying water was prepared in terms of the amount of water added. The aforementioned “smoothness during application”, “reduced coarse/rough feeling of hair after drying”, “smoothness of finger combing after drying”, and “viscosity” were measured and evaluated for the obtained cosmetic compositions. Results are shown in Table 3.

TABLE 2
Compositions of cosmetic compositions (mass %)

| One of polymers (1) through (12) | | 1 |
|---|---|---|
| Cationic surfactant | Stearyl trimethyl ammonium chloride | 1.5 |
| Higher alcohol | Cetyl alcohol | 2 |
| | Stearyl alcohol | 3 |
| Silicone oil | Polydimethyl siloxane | 1.5 |
| Hydrocarbon oil | Isoparaffin | 2 |
| Thickening agent | Hydroxyethyl cellulose | 0.5 |
| Humectant | Glycerin | 5 |
| pH adjustment agent | Citric acid | 0.02 |
| Preservative | Sodium benzoate | 0.2 |

| | |
|---|---|
| Water | Balance |

Stearyl trimethyl ammonium chloride: Wako Pure Chemical Industries
Cetyl alcohol: Wako Pure Chemical Industries
Stearyl alcohol: Wako Pure Chemical Industries
Polydimethyl siloxane: Dow Corning Toray Silicone Co., Ltd. “SH200”

### Isoparaffin: Idemitsu Kosan Co., Ltd. “IP2028”

Hydroxyethyl cellulose: Wilbur-Ellis Co. “Natrosol 250HR
Glycerin: Tokai Seiyaku “Japanese Pharmacopeia glycerin”

TABLE 3
Evaluation results for cosmetic compositions

| | Working Example | | | | | | | | Comparative Example | | |
|---|---|---|---|---|---|---|---|---|---|---|---|
| | 1-1 | 1-2 | 1-3 | 1-4 | 1-5 | 1-6 | 1-7 | 1-8 | 1-1 | 1-2 | 1-3 |
| Polymer used | Copoly- | Copoly- | Copoly- | Copoly- | Copoly- | Copoly- | Copoly- | Copoly- | Copoly- | Copoly- | Copoly- |
| | mer (1) | mer (2) | mer (3) | mer (4) | mer (5) | mer (6) | mer (7) | mer (8) | mer (9) | mer (10) | mer (11) |
| Smoothness | +1 | +1 | +1 | 0 | +1 | +1 | +1 | +1 | +1 | 0 | 0 |
| during | | | | | | | | | | | |
| application | | | | | | | | | | | |
| Reduced | +2 | +1 | +2 | +1 | +2 | +1 | +1 | +2 | 0 | 0 | 0 |
| coarse/rough | | | | | | | | | | | |
| feeling of hair | | | | | | | | | | | |
| after drying | | | | | | | | | | | |
| Smoothness | +2 | +2 | +2 | +2 | +1 | +1 | 0 | +1 | +1 | +1 | 0 |
| of finger | | | | | | | | | | | |
| combing after | | | | | | | | | | | |
| drying | | | | | | | | | | | |
| Viscosity | 6850 | 8000 | 2600 | 1200 | 16000 | 9750 | 22500 | 21250 | 6300 | 400 | 1300 |
| (mPa · s) | | | | | | | | | | | |

### [Evaluation of Results (1)]

(1-1) Whereas the copolymer used in the cosmetic composition according to comparative example 1-3 had a constituent unit derived from the vinyl monomer (A) beneath the range of the present invention at 30 mass %, the copolymers used in the cosmetic compositions of working examples 1-1 to 1-8 comprised at least 50 mass % of the constituent unit derived from the vinyl monomer (A) including the monomer (a), with there being no loss of smoothness during application, no coarse/rough feeling of hair after drying, and good smoothness of finger combing after drying.

(1-2) The copolymers used in the cosmetic compositions of working examples 1-5 had constituent units derived from the vinyl monomer represented by formula (3) as a highly hydrophobic monomer (b), and the copolymers used in the cosmetic composition of working examples 1-8 had highly hydrophobic cyclohexyl methacrylates as constituent units derived from, monomer (d), and thus were satisfactory, showing no coarse/rough feeling of hair after drying.

(1-3) The copolymers used in the cosmetic compositions of working examples 1-1 and 1-3 had constituent units derived from (meth)acrylic ester monomers having short polyethylene glycol chains, which had especially high hydrophobic properties among the different monomers (c); both exhibited no coarse/rough feeling of hair after drying, and had good smoothness of finger combing.

(1-4) Whereas the copolymer used in the cosmetic composition of comparative example 1 was a polymer comprising only the monomer (a), the copolymers used in the cosmetic compositions of working examples 1-1 to 1-8 were copolymers with other monomers; all showed a superiority in reduced coarse/rough feeling of hair after drying.

(1-5) Whereas the cosmetic composition according to comparative example 1-2 was a copolymer not comprising a constituent unit derived from any of monomers (b), (c), or (d), the copolymer used in the cosmetic compositions of working examples 1-1 to 1-8 were copolymers comprising a constituent unit derived from one of monomers (b) to (d), and thus had superior smoothness during application and reduced coarse/rough feeling of hair after drying.

### Working Examples 2-1 to 2-9, Comparative Example 2-1

Using polymers (1) to (6), (8), (11), and (12), cosmetic compositions having the compositions shown in Table 4 were prepared according to a normal method. Masses for each ingredient are in terms of active ingredients, and the accompanying water was prepared in terms of the amount of water added. The aforementioned “smoothness during application”, “smoothness of finger combing after drying”, and “hair manageability after drying” were measured and evaluated for the obtained cosmetic compositions. Results are shown in Table 5.

TABLE 4
Compositions of cosmetic compositions (mass %)

| One of polymers (1) through (12) | | 0.3 |
|---|---|---|
| Cationic surfactant | Stearyl trimethyl ammonium chloride | 1.5 |
| Higher alcohol | Cetyl alcohol | 2 |
| | Stearyl alcohol | 3 |
| Thickening agent | Hydroxyethyl cellulose | 0.5 |
| Humectants | Glycerin | 5 |
| pH adjustment agent | Citric | 0.02 |
| Preservative | Sodium benzoate | 0.2 |

| | |
|---|---|
| Water | Balance |

Stearyl trimethyl ammonium chloride: Wako Pure Chemical Industries
Cetyl alcohol: Wako Pure Chemical Industries
Stearyl alcohol: Wako Pure Chemical Industries
Isoparaffin: Idemitsu Kosan Co., Ltd. “IP2028”
Hydroxyethyl cellulose: Wilbur-Ellis Co. “Natrosol 250HR
Glycerin: Tokai Seiyaku “Japanese Pharmacopeia glycerin”

TABLE 5
Evaluation results for cosmetic compositions

| | | | | | | | | | Comparative |
|---|---|---|---|---|---|---|---|---|---|
| | Working Example | | | | | | | | Example |
| | 2-1 | 2-2 | 2-3 | 2-4 | 2-5 | 2-6 | 2-7 | 2-8 | 2-1 |
| Polymer used | Copolymer | Copolymer | Copolymer | Copolymer | Copolymer | Copolymer | Copolymer | Copolymer | Copolymer |
| | (1) | (2) | (3) | (4) | (5) | (6) | (8) | (12) | (11) |
| Smoothness during | +1 | +1 | +1 | 0 | +1 | +1 | +1 | +1 | 0 |
| application | | | | | | | | | |
| Smoothness of | +1 | +2 | +1 | +1 | +1 | +1 | +1 | +2 | 0 |
| finger combing | | | | | | | | | |
| after drying | | | | | | | | | |
| Hair manageability | 0 | +2 | 0 | 0 | +1 | +1 | +1 | +1 | 0 |
| after drying | | | | | | | | | |

### [Evaluation of Results (2)]

(2-1) Whereas the copolymer used in the cosmetic composition according to comparative example 2-1 had a constituent unit derived from the vinyl monomer (A) beneath the range of the present invention at 30 mass %, the copolymers used in the cosmetic compositions of working examples 2-1 to 2-8 comprised at least 50 mass % of the constituent unit derived from the vinyl monomer (A) including the monomer (a), with there being no loss of smoothness during application, no coarse/rough feeling of hair after drying, and good smoothness of finger combing after drying.

(2-2) The copolymers used in the cosmetic compositions according to working example 2-2 and 2-8 comprised a greater proportion of monomer (a) than the other working examples and comparative examples, and these examples therefore exhibited especially good smoothness of finger combing after drying.

### Working Examples/Comparative Examples of Cosmetic Composition According to the Second Aspect of the Present Invention

Working examples and comparative examples of the cosmetic composition according to the first aspect of the present invention will be described below.

### Preparation/Evaluation of Cosmetic Compositions

### Working Examples 3-1 to 3-8 and Comparative Examples 3-1 to 3-3

Using copolymers (1) to (12), cosmetic compositions having the compositions shown in Table 2, “Working examples/comparative examples for cosmetic compositions according to the first aspect of the present invention” were prepared according to a normal method. Masses for each ingredient are in terms of active ingredients, and the accompanying water was prepared in terms of the amount of water added. The aforementioned “smoothness during application”, “reduced coarse/rough feeling of hair after drying”, “smoothness of finger combing after drying”, “viscosity”, and “silicone oil adsorption” were measured and evaluated for the obtained cosmetic compositions. Results are shown in Table 6.

TABLE 6
Evaluation results for cosmetic compositions

| | | | | | | | | | | | Comparative |
|---|---|---|---|---|---|---|---|---|---|---|---|
| | Working Example | | | | | | | | | | Example |
| | 3-1 | 3-2 | 3-3 | 3-4 | 3-5 | 3-6 | 3-7 | 3-8 | 3-9 | 3-10 | 3-1 |
| Polymer used | Copoly- | Copoly- | Copoly- | Copoly- | Copoly- | Copoly- | Copoly- | Copoly- | Copoly- | Copoly- | Copolymer |
| | mer (1) | mer (2) | mer (3) | mer (4) | mer (5) | mer (6) | mer (7) | mer (8) | mer (9) | mer (10) | (11) |
| Smoothness | +1 | +1 | +1 | 0 | +1 | +1 | +1 | +1 | +1 | 0 | 0 |
| during | | | | | | | | | | | |
| application | | | | | | | | | | | |
| Reduced | +2 | +1 | +2 | +1 | +2 | +1 | +1 | +2 | 0 | 0 | 0 |
| coarse/rough | | | | | | | | | | | |
| feeling of hair | | | | | | | | | | | |
| after drying | | | | | | | | | | | |
| Smoothness | +2 | +2 | +2 | +2 | +1 | +1 | 0 | +1 | +1 | +1 | 0 |
| of finger | | | | | | | | | | | |
| combing after | | | | | | | | | | | |
| drying | | | | | | | | | | | |
| Viscosity | 6850 | 8000 | 2600 | 1200 | 16000 | 9750 | 22500 | 21250 | 6300 | 400 | 1300 |
| (mPa · s) | | | | | | | | | | | |
| Silicone oil | 860 | 1500 | 1800 | 900 | 560 | 710 | 255 | 700 | 1250 | 1300 | 250 |
| adsorption | | | | | | | | | | | |
| (wtppm) | | | | | | | | | | | |

### [Evaluation of Results (3)]

(3-1) Whereas the copolymer used in the cosmetic composition according to comparative example 3-1 had a constituent unit derived from the vinyl monomer (A) beneath the range of the present invention at 30 mass %, the copolymers used in the cosmetic compositions of working examples 3-1 to 3-10 comprised at least 50 mass % of the constituent unit derived from the vinyl monomer (A) including the monomer (a), with there being no loss of smoothness during application, no coarse/rough feeling of hair after drying, and good smoothness of finger combing after drying.

(3-2) The copolymer used in the cosmetic composition of working examples 3-5 had a constituent unit derived from the vinyl monomer represented by formula (3) as a highly hydrophobic monomer (b), and the copolymer used in the cosmetic composition of working example 1-8 had highly hydrophobic cyclohexyl methacrylate as a constituent unit derived from monomer (d); both were satisfactory, showing no coarse/rough feeling of hair after drying.

(3-3) The copolymers used in the cosmetic compositions of working examples 3-1 and 3-3 had constituent units derived from (meth)acrylic ester monomers having short polyethylene glycol chains, which had especially high hydrophobic properties among the different monomers (c); both exhibited no coarse/rough feeling of hair after drying, and had good smoothness of finger combing after drying.

(3-4) Out of working examples 3-1 to 3-10, those showing high levels of silicone oil adsorption had good smoothness of finger combing after drying.

### Working Examples 4-1 to 4-9, Comparative Example 4-1

Using copolymers (1) to (6), (8), (11), and (12), cosmetic compositions having the compositions shown in Table 4. “Working examples/comparative examples for cosmetic compositions according to the first aspect of the present invention” were prepared according to a normal method. Masses for each ingredient are in terms of active ingredients, and the accompanying water was prepared in terms of the amount of water added. The aforementioned “smoothness during application”, “smoothness of finger combing after drying”, and “hair manageability after drying” were measured and evaluated for the obtained cosmetic compositions. Results are shown in Table 7.

TABLE 7
Evaluation results for cosmetic compositions

| | Working | Working | Working | Working | Working | Working | Working | Working | Comparative |
|---|---|---|---|---|---|---|---|---|---|
| | Example | Example | Example | Example | Example | Example | Example | Example | Example |
| | 4-1 | 4-2 | 4-3 | 4-4 | 4-5 | 4-6 | 4-7 | 4-8 | 4-1 |
| Polymer used | Copoly- | Copolymer | Copolymer | Copolymer | Copolymer | Copolymer | Copolymer | Copolymer | Copolymer |
| | mer (1) | (2) | (3) | (4) | (5) | (6) | (8) | (12) | (11) |
| Smoothness during | +1 | +1 | +1 | 0 | +1 | +1 | +1 | +1 | 0 |
| application | | | | | | | | | |
| Smoothness of finger | +1 | +2 | +1 | +1 | +1 | +1 | +1 | +2 | 0 |
| combing after drying | | | | | | | | | |
| Hair mangeability | 0 | +2 | 0 | 0 | +1 | +1 | +1 | +1 | 0 |
| after drying | | | | | | | | | |

### [Evaluation of Results (4)]

(4-1) Whereas the copolymer used in the cosmetic composition according to comparative example 4-1 had a constituent unit derived from the vinyl monomer (A) beneath the range of the present invention at 30 mass %, the copolymers used in the cosmetic compositions of working examples 4-1 to 4-8 comprised at least 50 mass % of the constituent unit derived from the vinyl monomer (A) including the monomer (a), with there being no loss in smoothness during application, good smoothness of finger combing after drying, and good manageability after drying.

(4-2) The cosmetic compositions according to working example 4-2 and 4-8 comprised a greater proportion of monomer (a) than the other working examples and comparative examples, and these examples therefore exhibited especially good smoothness of finger combing after drying.

### INDUSTRIAL APPLICABILITY

The cosmetic composition according to the present invention enables superior conditioning effects to be obtained, particularly when used as a hair cosmetic. For this reason, the cosmetic composition is especially suited for use as a conditioning agent.

The dimensions and values disclosed herein are not to be understood as being strictly limited to the exact numerical values recited. Instead, unless otherwise specified, each such

dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition comprising:
from 0.05 to 5 mass % of a copolymer (α);
from 0.1 to 20 mass % of a cationic surfactant; and
from 0.1 to 20 mass % of a higher alcohol;
wherein the copolymer (α) includes a constituent unit derived from a monomer (a) below and at least one constituent unit derived from monomers (b) to (d) below;
wherein the copolymer (α) comprises from 1 to 99 mass % of the constituent unit derived from the monomer (a) below per a total mass of the copolymer (α), and from 50 to 100 mass % of the constituent unit derived from vinyl monomer (A) having a carboxyl group;
and wherein the copolymer (α) has a mass average molecular weight of from 3,000 to 400,000;
wherein the Monomer (a) is acrylic acid and/or methacrylic acid;
wherein the Monomer (b) is one or two or more types of vinyl monomers expressed by formulas (1), (2), and (3) below:
CH₂═C(R¹)—CO—(O—(CH₂)ₘ—CO)ₙ—OH (1)
(in formula (1), R¹ is a hydrogen atom or a methyl group, “m” is an integer from 1 to 4, and “n” is an integer from 1 to 4);
CH₂═C(R²)—COO—(CH₂)ₚ—OCO—(CH₂)_{q}—COOH (2)
(in formula (2), R² is a hydrogen atom or a methyl group, and “p” and “q” are each independently an integer from 2 to 6);
CH₂═C(R³)—COO—(CH₂)ₑ—OCO—X—COOH (3)
(in formula (3), R³ is a hydrogen atom or a methyl group, “e” is an integer from 1 to 4, and “X” is a cyclohexylene group optionally having a substituent or a phenylene group optionally having a substituent);
wherein the Monomer (c) is a vinyl monomer expressed by formula (4) below:
CH₂═C(R⁴)—CO—Y—(Z—O)ᵣ—R⁵ (4)
(in formula (4), R⁴ is a hydrogen atom or a methyl group, R⁵ is a hydrogen atom or an alkyl group having from 1 to 5 carbons, “Y” is an oxygen atom or NH, “Z” is an alkylene group having from 2 to 4 carbons and optionally having a substituent, and “r” is an integer from 1 to 15); and
wherein the Monomer (d) is a vinyl monomer expressed by formula (5) below:
CH₂═C(R⁶)—COO—R⁷ (5)
(in formula (5), R⁶ is a hydrogen atom or a methyl group, R⁷ is a straight chain or branched alkyl group having from 12 to 22 carbons, a cyclohexyl group optionally having a substituent, or a phenyl group optionally having a substituent).

2. The cosmetic composition according to claim 1, wherein the copolymer (α) comprises from 1 to 99 mass % of the constituent unit derived from the monomer (b) per the total mass of the copolymer (α).

3. The cosmetic composition according to claim 1 or 2, wherein the copolymer (α) comprises from 1 to 50 mass % of the constituent unit derived from the monomer (c) per the total mass of the copolymer (α).

4. The cosmetic composition according to any one of claims 1 to 3, wherein the copolymer (α) comprises from 1 to 50 mass % of the constituent unit derived from the monomer (d) per the total mass of the copolymer (α).

5. The cosmetic composition according to any one of claims 1 to 4, wherein the constituent unit derived from the vinyl monomer (A) is comprised at a ratio of greater than 60 mass % but not greater than 99 mass % per the total mass of the copolymer (α).

6. The cosmetic composition according to any one of claims 1 to 5, further comprising from 0.1 to 10 mass % of a silicone oil.

7. The cosmetic composition according to any one of claims 1 to 5, having a silicone oil content of less than 0.1 mass %

8. A conditioning agent comprising the cosmetic composition described in any one of claims 1 to 7.

9. A cosmetic composition comprising:
from 0.05 to 5 mass % of a copolymer ((3);
from 0.1 to 20 mass % of a cationic surfactant; and
from 0.1 to 20 mass % of a higher alcohol;
wherein the copolymer (β) includes a constituent unit derived from a monomer (a) below and at least one constituent unit derived from monomers (b) to (e) below;
wherein the copolymer (β) comprises from 50 to 100 mass % of the constituent unit derived from vinyl monomer (A) having a carboxyl group per a total mass of the copolymer (β), and wherein from 50 to 100 mol % of the carboxyl groups are not neutralized;
and wherein the copolymer (β) has a mass average molecular weight of from 3,000 to 400,000;
wherein the Monomer (a) is acrylic acid and/or methacrylic acid;
wherein the Monomer (b) is one or two or more types of vinyl monomers expressed by formulas (1), (2), and (3) below:
CH₂═C(R¹)—CO—(O—(CH₂)ₘ—CO)ₙ—OH (1)
(in formula (1), R¹ is a hydrogen atom or a methyl group, “m” is an integer from 1 to 4, and “n” is an integer from 1 to 4);
CH₂═C(R²)—COO—(CH₂)ₚ—OCO—(CH₂)_{q}—COOH (2)
(in formula (2), R² is a hydrogen atom or a methyl group, and “p” and “q” are each independently an integer from 2 to 6);
CH₂═C(R³)—COO—(CH₂)ₑ—OCO—X—COOH (3)
(in formula (3), R³ is a hydrogen atom or a methyl group, “e” is an integer from 1 to 4, and “X” is a cyclohexylene group optionally having a substituent or a phenylene group optionally having a substituent);
wherein the Monomer (c): a vinyl monomer expressed by formula (4) below:
CH₂═C(R⁴)—CO—Y—(Z—O)ᵣ—R⁵ (4)
(in formula (4), R⁴ is a hydrogen atom or a methyl group, R⁵ is a hydrogen atom or an alkyl group having from 1 to 5 carbons, “Y” is an oxygen atom or NH, “Z” is an alkylene group having from 2 to 4 carbons and optionally having a substituent, and “r” is an integer from 1 to 15);
wherein the Monomer (d): a vinyl monomer expressed by formula (5) below:
CH₂═C(R⁶)—COO—R⁷ (5)
(in formula (5), R⁶ is a hydrogen atom or a methyl group, R⁷ is a straight chain or branched alkyl group having from 12 to 22 carbons, a cyclohexyl group optionally having a substituent, or a phenyl group optionally having a substituent), and wherein the Monomer (e): a vinyl monomer expressed by formula (6) below:
CH₂═C(R⁸)—CO—N(R⁹)R¹⁰ (6)
(in formula (6), R8 is a hydrogen atom or a methyl group, R9 and R10 are each independently a hydrogen atom or a straight chain or branched alkyl group having from 1 to 4 carbons).

10. The cosmetic composition according to claim 9, wherein the copolymer (β) comprises from 1 to 99 mass % of the constituent unit derived from the monomer (b) per the total mass of the copolymer (β).

11. The cosmetic composition according to claim 9 or 10, wherein the copolymer (β) comprises from 1 to 50 mass % of the constituent unit derived from the monomer (c) per the total mass of the copolymer (β).

12. The cosmetic composition according to any one of claims 9 to 11, wherein the copolymer (β) comprises from 1 to 50 mass % of the constituent unit derived from the monomer (d) per the total mass of the copolymer (β).

13. The cosmetic composition according to any one of claims 9 to 12, wherein the copolymer (β) comprises from 1 to 50 mass % of the constituent unit derived from the monomer (e).

14. The cosmetic composition according to any one of claims 9 to 13, wherein the constituent unit derived from the vinyl monomer (A) is comprised at a ratio of greater than 60 mass % but not greater than 99 mass % per the total mass of the copolymer (β).

15. The cosmetic composition according to any one of claims 9 to 14, further comprising from 0.1 to 10 mass % of a silicone oil.

16. The cosmetic composition according to any one of claims 9 to 15, having a silicone oil content of less than 0.1 mass %.

17. A conditioning agent comprising the cosmetic composition according to any one of claims 9 to 16.
